(19) United States
(12) Patent Application Publication
(10) Pub. No.: US 2025/0010031 A1
(43) Pub. Date: Jan. 9, 2025

(54) CATHETER

(71) Applicant: KANEKA CORPORATION, Osaka-shi (JP)

(72) Inventor: Kazuaki IKOMA, Settsu-shi (JP)

(73) Assignee: KANEKA CORPORATION, Osaka-shi (JP)

(21) Appl. No.: 18/891,829

(22) Filed: Sep. 20, 2024

Related U.S. Application Data
(63) Continuation of application No. PCT/JP2023/009132, filed on Mar. 9, 2023.

(30) Foreign Application Priority Data
Mar. 29, 2022 (JP) 2022-054634
Sep. 25, 2023 (JP) 2023-160011
Sep. 25, 2023 (JP) 2023-160012

(51) Int. Cl. A61M 25/00

(52) U.S. Cl. CPC A61M 25/0043; A61M 25/0026; A61M 2205/02

(57) ABSTRACT

A catheter includes: an outer tube; a bag-shaped body provided to a distal portion of the outer tube, the bag-shaped body having an annular shape and having a portion that has a smaller outer diameter than the outer tube; and a flow path configured to allow a fluid to be injected therethrough to the bag-shaped body, wherein the bag-shaped body is configured so that a minimum inner diameter of the bag-shaped body in a pressurized state where the bag-shaped body is pressurized by injecting a fluid into the flow path is larger than a minimum inner diameter of the bag-shaped body in a non-pressurized state where the bag-shaped body is not pressurized by injecting a fluid into the flow path.

### CROSS REFERENCE TO RELATED APPLICATIONS

This application is a Continuation of PCT International Application No. PCT/JP2023/009132, filed on Mar. 9, 2023, which claims priority under 35 U.S.C. 119 (a) to Patent Application No. 2022-054634, filed in Japan on Mar. 29, 2022, Patent Application No. 2023-160011, filed in Japan on Sep. 25, 2023, Patent Application No. 2023-160012, filed in Japan on Sep. 25, 2023, all of which are hereby expressly incorporated by reference into the present application.

### TECHNICAL FIELD

The present invention relates to a catheter.

### BACKGROUND ART

For delivering a medical implement such as a balloon or a stent to a treatment site which is a treatment target, a resin tube is used, for example. Specifically, the resin tube is, in a state where the medical implement is disposed inside a lumen of the resin tube, inserted into a body cavity such as a blood vessel and delivered to the treatment site. In a case where the resin tube used at this time has a simple tubular shape, contact of a tip portion thereof with the wall of the body cavity leads to problems that: the wall of the body cavity is damaged; and passability deteriorates.

Patent Document 1 describes a catheter that can make it less likely to injure the wall of a body cavity. This catheter has a long elastic tubular part having at least one lumen. This catheter has a distal end to which a soft deformable tip part is attached. The tip part is pressed against a comparatively stationary surface so as to have an increased outer diameter, whereby the contact area is increased. Patent Document 1 describes that the increase in the contact area makes it possible to reduce the pressure or force, per unit area, to be applied to a tissue.

Patent Document 2 describes a medical technical instrument having favorable operability. This instrument has: an elongated deformable internal body having a tubular shape; an elongated outer covering body located on a radially outer side and at least partially enclosing the internal body; and a device for changing the state of this instrument from a deformable state to a stiffened state and from the stiffened state to the deformable state. The internal body is formed in the form of a double tube having: an inner tube forming an inner wall; and an outer tube forming an outer wall and concentrically enclosing the inner tube. Patent Document 2 describes that: the device for changing the state of this instrument from the deformable state to the stiffened state and from the stiffened state to the deformable state increases the internal pressure of an annular gap so as to cause the outer tube to bulge in a radial direction so that a pressure is applied to the covering body so as to change the state of this instrument to the stiffened state; and this instrument is easily operated. Patent Document 2 further describes that, although the outer diameter of the double tube of the internal body is increased, the size of the inner space of the internal body is kept unchanged.

### PRIOR ART DOCUMENTS

### Patent Documents

Patent Document 1: JP-A-60-040069
Patent Document 2: JP-T-2009-505700

### SUMMARY OF THE INVENTION

### Problems to be Solved by the Invention

However, the catheter described in Patent Document 1 has the following drawback. That is, this catheter increases the outer diameter of the tip part, thereby preventing a body cavity from being damaged. Consequently, in the configuration of the catheter described in Patent Document 1, the diameter-increased tip portion easily gets caught on the wall of a body cavity. Thus, this catheter cannot solve the problem that the passability in a body cavity is poor.

In addition, the medical technical instrument described in Patent Document 2 also has the following drawback. That is, this instrument applies a pressure so as to increase the outer diameter, thereby obtaining the stiffened state and improving operability. Consequently, the diameter-increased portion easily gets caught on the internal wall of a body cavity. Thus, there is still room for enhancement in terms of improvement of the passability in a body cavity.

In addition, in each of the catheter in Patent Document 1 and the medical technical instrument in Patent Document 2, an inner diameter with a value that allows passage of a medical implement such as a balloon or a stent needs to be ensured in order to place the medical implement at a treatment site which is a treatment target after delivering the medical implement to the treatment site.

The present invention has been made in view of the aforementioned circumstances, and an object of the present invention is to provide a catheter that can make it easy to prevent the wall of a body cavity from being damaged when the catheter is delivered to a treatment site and that can also make it easy to improve the passability in a body cavity.

### Solutions to the Problems

An embodiment of the present invention that can overcome the above problems is, for example, any of the following [1] to [22].
[1] A catheter including: an outer tube having a distal end and a proximal end and having a lumen extending in a longitudinal direction; a bag-shaped body provided to a distal portion of the outer tube, the bag-shaped body having an annular shape and having a portion that has a smaller outer diameter than the outer tube; and a flow path configured to allow a fluid to be injected therethrough to the bag-shaped body, wherein the bag-shaped body is configured so that a minimum inner diameter of the bag-shaped body in a pressurized state where the bag-shaped body is pressurized by injecting a fluid into the flow path is larger than a minimum inner diameter of the bag-shaped body in a non-pressurized state where the bag-shaped body is not pressurized by injecting a fluid into the flow path.
[2] The catheter according to above [1], wherein the bag-shaped body is further configured so that in the non-pressurized state of the bag-shaped body, the bag-shaped body has an inner surface portion facing an inner side and an outer surface portion facing an outer side, and when a state of the bag-shaped body is changed from the non-pressurized state to the pressurized state, an elongation rate of a length from a distal end to a proximal end of the inner surface portion is higher than an elongation rate of a length from a distal end to a proximal end of the outer surface portion.
[3] The catheter according to above [2], wherein, in at least one of the non-pressurized state of the bag-shaped body and the pressurized state of the bag-shaped body, an angle formed between inner walls of the bag-shaped body in a cross section parallel to the longitudinal direction is an acute angle.
[4] The catheter according to above [2] or [3], wherein the bag-shaped body and the outer tube are further configured so that when the state of the bag-shaped body is changed from the non-pressurized state to the pressurized state, the elongation rate of the length from the distal end to the proximal end of the inner surface portion is higher than an elongation rate of a length from the distal end to the proximal end of the outer tube.
[5] The catheter according to any one of above [1] to [4], wherein the bag-shaped body is disposed on a distal side relative to the distal end of the outer tube and has a tapered portion having an outer diameter that is reduced toward the distal side.
[6] The catheter according to any one of above [1] to [5], wherein the catheter further includes a radiopaque marker, wherein the radiopaque marker is provided to at least one of the bag-shaped body and a region, of the outer tube, that extends 10 cm toward a proximal side from the distal end of the outer tube.
[7] The catheter according to any one of above [1] to [6], wherein the outer tube has, at a proximal portion of the outer tube, a reduced diameter area having a diameter that is reduced toward a proximal side in a cross-sectional view perpendicular to the longitudinal direction.
[8] The catheter according to above [7], wherein the catheter further includes a first tubular member having a maximum outer diameter smaller than a maximum outer diameter of the outer tube, the first tubular member having a lumen extending in the longitudinal direction, the first tubular member has a distal portion fixed to a proximal portion of the reduced diameter area of the outer tube, and the lumen of the first tubular member and the flow path are in communication with each other.
[9] The catheter according to any one of above [1] to [8], wherein, in the pressurized state, the bag-shaped body has an outer diameter equal to or smaller than an outer diameter of the outer tube.
[10] The catheter according to any one of above [1] to [9], wherein the catheter further includes at least one bulging portion configured to bulge inward in a radial direction of the outer tube or outward in the radial direction of the outer tube, and the bulging portion and the flow path are configured to allow a fluid to be injected to the bulging portion though the flow path.
[11] The catheter according to above [10], wherein the at least one bulging portion is configured to bulge inward in the radial direction of the outer tube when the at least one bulging portion is pressurized at a first predetermined pressure or higher pressure by injecting a fluid into the flow path, the bag-shaped body is configured so that a minimum inner diameter of the bag-shaped body in a state where the bag-shaped body is pressurized at a second predetermined pressure or higher pressure by injecting a fluid into the flow path is larger than the minimum inner diameter of the bag-shaped body in the non-pressurized state, and the second predetermined pressure is lower than the first predetermined pressure.
[12] The catheter according to above [10], wherein the at least one bulging portion is configured to bulge outward in the radial direction of the outer tube when the at least one bulging portion enters a state of being pressurized at a first predetermined pressure or higher pressure by injecting a fluid into the flow path, the bag-shaped body is configured so that a minimum inner diameter of the bag-shaped body in a state where the bag-shaped body is pressurized at a second predetermined pressure or higher pressure by injecting a fluid into the flow path is larger than the minimum inner diameter of the bag-shaped body in the non-pressurized state, and the second predetermined pressure is equal to or higher than the first predetermined pressure.
[13] The catheter according to any one of above to [12], wherein the flow path, the bag-shaped body, and the bulging portion are configured so that a fluid injected is allowed to flow to the bag-shaped body passes through the flow path and the bulging portion.
[14] The catheter according to any one of above to [13], wherein the at least one bulging portion has a first inward bulging portion and a second inward bulging portion configured to bulge inward in the radial direction of the outer tube and arranged at positions that mutually differ in a circumferential direction, and the first inward bulging portion and the second inward bulging portion are configured so that in the pressurized state, the first inward bulging portion comes into contact with the second inward bulging portion.
[15] The catheter according to above [10], wherein, in the pressurized state, the at least one bulging portion bulges inward in the radial direction of the outer tube, to close a portion of the lumen of the outer tube.
[16] The catheter according to any one of above to [15], wherein, in the pressurized state, a bulging height of the at least one bulging portion in the radial direction of the outer tube varies according to a position of the outer tube in a circumferential direction.
[17] The catheter according to any one of above to [16], wherein the at least one bulging portion has a first bulging portion and a second bulging portion arranged at positions that mutually differ in a circumferential direction, and a bulging height of the first bulging portion and a bulging height of the second bulging portion differ from each other.
[18] The catheter according to above [10], wherein the at least one bulging portion bulges inward in the radial direction of the outer tube, and a frictional force of a surface of the at least one bulging portion located on an inner side in the radial direction of the outer tube is higher than a frictional force of an inner surface, of the outer tube, that faces the lumen of the outer tube.
[19] The catheter according to above [10], wherein the at least one bulging portion bulges outward in the radial direction of the outer tube, and a frictional force of an outer surface of the at least one bulging portion is higher than a frictional force of an outer surface of the outer tube.
[20] The catheter according to above [10], wherein the at least one bulging portion has an outward bulging portion configured to bulge outward in the radial direction of the outer tube in the pressurized state, and the at least one bulging portion is located on a proximal side relative to a proximal end of the bag-shaped body.
[21] A catheter including: an outer tube having a distal end and a proximal end and having a lumen extending in a longitudinal direction, the outer tube having a hollow portion at a distal portion, wherein the hollow portion annularly extends within a wall defining the outer tube; and a flow path configured to allow a fluid to be injected therethrough to the hollow portion, wherein the outer tube is configured so that a minimum inner diameter at a distal end portion of the outer tube in a pressurized state where the hollow portion is pressurized by injecting a fluid into the flow path is larger than a minimum inner diameter at the distal end portion of the outer tube in a non-pressurized state where the hollow portion is not pressurized by injecting a fluid into the flow path.
[22] A catheter including: an outer tube having a distal end and a proximal end and having a lumen extending in a longitudinal direction, the outer tube having a hollow portion at a distal portion, wherein the hollow portion annularly extends within a wall defining the outer tube; and a flow path configured to allow a fluid to be injected therethrough to the hollow portion, wherein the outer tube is configured so that an inner diameter of the outer tube at a distal end portion is enlarged by injecting a fluid into the flow path to apply pressure to the follow portion.

### Effects of Invention

According to the catheter, the wall of the body cavity is easily prevented from being damaged by the catheter, and the passability of the catheter in the body cavity is also easily improved.

### BRIEF DESCRIPTION OF THE DRAWINGS

FIG. 1 is a side view showing an example of a catheter according to an embodiment of the present invention.

FIG. 2 is an enlarged cross-sectional view of the distal side of the catheter shown in FIG. 1.

FIG. 3 is an end view, taken along the line III-III, of the catheter shown in FIG. 2.

FIG. 4 is an enlarged cross-sectional view of a distal side of the catheter shown in FIG. 1 and shows a non-pressurized state where pressurization by injecting a fluid into a flow path is not performed.

FIG. 5 is an end view, taken along the line V-V, of the catheter shown in FIG. 4.

FIG. 6 is a cross-sectional view of the catheter shown in FIG. 4 when the catheter enters a pressurized state of being pressurized by injecting a fluid into the flow path.

FIG. 7 is a cross-sectional view showing a modification of the catheter shown in FIG. 4.

FIG. 8 is an end view, taken along the line VIII-VIII, of the catheter shown in FIG. 7.

FIG. 9 is a cross-sectional view showing another modification of the catheter shown in FIG. 4.

FIG. 10 is an end view, taken along the line X-X, of the catheter shown in FIG. 9.

FIG. 11 is a cross-sectional view showing another modification of the catheter shown in FIG. 4.

FIG. 12 is an end view, taken along the line XII-XII, of the catheter shown in FIG. 11.

FIG. 13 is a cross-sectional view (partial side view) of the catheter shown in FIG. 11 when the catheter enters a pressurized state of being pressurized by injecting a fluid into the flow path.

FIG. 14 is a cross-sectional view (partial side view) showing a modification of the catheter shown in FIG. 4.

FIG. 15 is an end view, taken along the line XV-XV, of the catheter shown in FIG. 14.

FIG. 16 is a cross-sectional view (partial side view) of the catheter shown in FIG. 14 when the catheter enters a pressurized state of being pressurized by injecting a fluid into the flow path.

FIG. 17 is a cross-sectional view of an example of a catheter according to another embodiment of the present invention and shows a pressurized state where the catheter is pressurized by injecting a fluid into a flow path.

FIG. 18 is a cross-sectional view, taken along the line XVIII-XVIII, of the catheter shown in FIG. 17.

FIG. 19 is a cross-sectional view (partial side view) of a modification of the catheter shown in FIG. 16 and shows a pressurized state where the catheter is pressurized by injecting a fluid into the flow path.

FIG. 20 is a cross-sectional view of an example of a catheter according to still another embodiment of the present invention and shows a state where a bulging portion is pressurized at a first predetermined pressure or higher pressure by injecting a fluid into a flow path.

FIG. 21 is a cross-sectional view showing a state where a bag-shaped body of the catheter shown in FIG. 20 is pressurized at a second predetermined pressure or higher pressure by injecting a fluid into the flow path.

FIG. 22 is a cross-sectional view, taken along the line XXII-XXII, of the catheter shown in FIG. 21.

FIG. 23 is a cross-sectional view of an example of a catheter according to still another embodiment of the present invention and shows a state where a bag-shaped body is pressurized by injecting a fluid into a flow path.

FIG. 24 is a cross-sectional view (partial side view) of a modification of the catheter shown in FIG. 23 and shows a pressurized state where the catheter is pressurized by injecting a fluid into the flow path.

FIG. 25 is a cross-sectional view showing a modification of the catheter shown in FIG. 18.

FIG. 26 is a cross-sectional view showing a modification of the catheter shown in FIG. 18.

FIG. 27 is a cross-sectional view showing a modification of the catheter shown in FIG. 22.

FIG. 28 is a cross-sectional view showing a modification of the catheter shown in FIG. 22.

FIG. 29 is a cross-sectional view showing a modification of the catheter shown in FIG. 4.

### DESCRIPTION OF EMBODIMENTS

The present invention will be specifically explained below based on the following embodiments, however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of the present invention.

One embodiment of a catheter according to the present invention includes: an outer tube having a distal end and a proximal end and having a lumen extending in a longitudinal direction; a bag-shaped body provided to a distal portion of the outer tube, the bag-shaped body having an annular shape and having a portion that has a smaller outer diameter than the outer tube; and a flow path configured to allow a fluid to be injected therethrough to the bag-shaped body, wherein a minimum inner diameter of the bag-shaped body in a pressurized state where the bag-shaped body is pressurized by injecting a fluid into the flow path is larger than a minimum inner diameter of the bag-shaped body in a non-pressurized state where the bag-shaped body is not pressurized by injecting a fluid into the flow path.

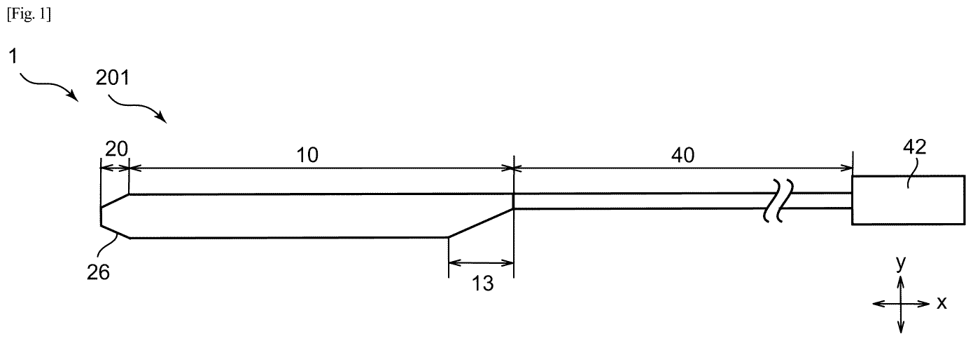

The overall structure of a catheter is explained with reference to FIGS. 1 to 29. For example, FIGS. 1, 2, 4, 6, 7, 9, 11, and 13 show a catheter 1 having: an outer tube 10; and a bag-shaped body 20 provided to a distal portion of the outer tube 10. In these drawings, a longitudinal direction of the outer tube 10 is represented by x, and a radial direction of the outer tube 10 is represented by y. The radial direction y is a direction perpendicular to the longitudinal direction x, and only one direction perpendicular to the longitudinal direction x is shown in the drawings. For facilitating understanding, a mode in which the longitudinal direction x of the outer tube 10 and a longitudinal direction of the catheter 1 are the same as each other, is shown in the drawings. However, the present invention is not limited to this mode.

In the description, a proximal side refers to a direction toward a user's hand with respect to an extending direction of the outer tube 10, and a distal side refers to the opposite direction from the proximal side, that is, the direction toward a treatment target side. A distal portion of each member refers to the distal half of the member, and a proximal portion of each member refers to the proximal half of the member. A distal end of each member or each portion refers to the most distal position of the member or the portion, and a proximal end of each member or each portion refers to the most proximal position of the member or the portion.

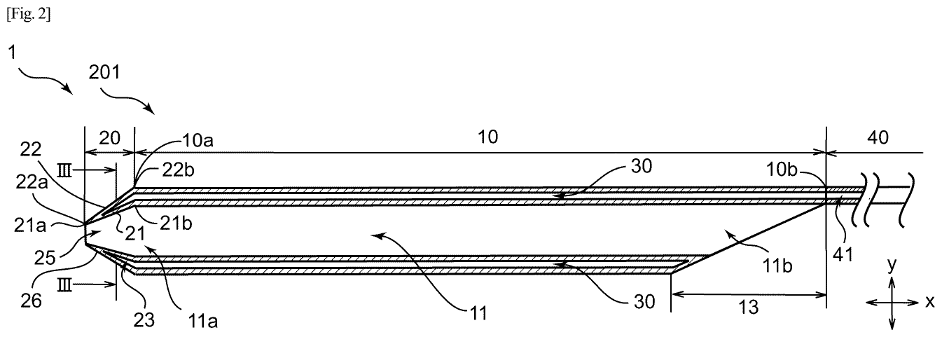

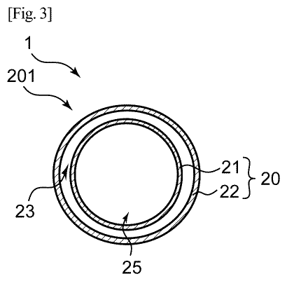

FIG. 1 is a side view showing an example of the catheter according to the embodiment of the present invention. FIG. 2 is an enlarged cross-sectional view of the distal side of the catheter 1 shown in FIG. 1. FIG. 3 is an end view, taken along the line III-III, of the catheter 1 shown in FIG. 2. FIGS. 1 to 3 each show a non-pressurized state where pressurization by injecting a fluid into a flow path is not performed.

As shown in FIGS. 1 and 2, the catheter 1 has the outer tube 10. The outer tube 10 has a distal end 10a and a proximal end 10b and has a lumen 11 extending in the longitudinal direction x. The lumen 11 is preferably in communication with the outside of the outer tube 10. A gap is preferably present inside the lumen 11 of the outer tube 10.

The outer diameter and/or the inner diameter of the outer tube 10 is preferably unchanged between before and after a fluid is injected into a flow path. As shown in FIGS. 1 and 2, the bag-shaped body 20 is provided to the distal portion of the outer tube 10. The bag-shaped body 20 is a portion having the shape of a bag. The bag-shaped body 20 is preferably located on the distal side relative to the distal end of the outer tube 10.

Since the bag-shaped body 20 has the shape of a bag, the bag-shaped body 20 preferably has an inner space 23 therein. The inner space 23 is preferably in communication with a flow path 30. With this configuration, a fluid injected into the flow path 30 can be caused to flow into the inner space 23 of the bag-shaped body 20 so as to pressurize the inside of the bag-shaped body 20. As a result, the inner diameter of the bag-shaped body 20 can be changed between a pressurized state where the bag-shaped body 20 is pressurized and a non-pressurized state where the bag-shaped body 20 is not pressurized. The pressurization preferably leads to deformation of the bag-shaped body 20 or expansion of the inner space 23.

As shown in FIGS. 1 to 3, the bag-shaped body 20 has an annular shape. The bag-shaped body 20 preferably has an annular shape by extending around a central axis in the longitudinal direction x. The bag-shaped body 20 preferably has an annular shape as seen in the longitudinal direction x. With the bag-shaped body 20 having an annular shape, the bag-shaped body 20 preferably has a lumen 25 located on an inner side of the annulus. The lumen 25 is preferably in communication with the outside of the bag-shaped body 20. The lumen 25 is preferably in communication with the lumen 11 of the outer tube 10. A gap is preferably present inside the lumen 25 of the bag-shaped body 20.

The bag-shaped body 20 has a portion that has a smaller outer diameter than the outer tube 10. An outer diameter of the portion, of the bag-shaped body 20, that has a smaller outer diameter than the outer tube 10 is defined in the non-pressurized state where the bag-shaped body 20 is not pressurized.

As shown in FIG. 2, the catheter 1 includes the flow path 30 which allows a fluid to be injected therethrough to the bag-shaped body 20. The flow path 30 is a space that, when a fluid is injected toward the bag-shaped body 20, allows the fluid to pass through the space. The flow path 30 functions as a path for supplying a fluid for pressurizing the bag-shaped body 20. The flow path 30 is preferably in communication with the inner space 23 of the bag-shaped body 20.

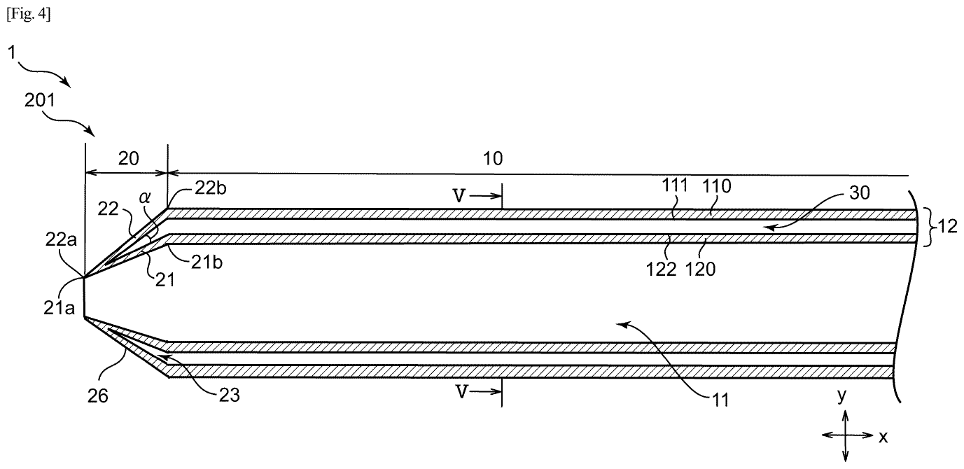

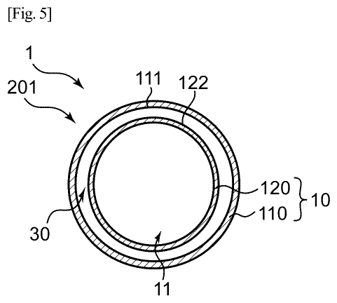

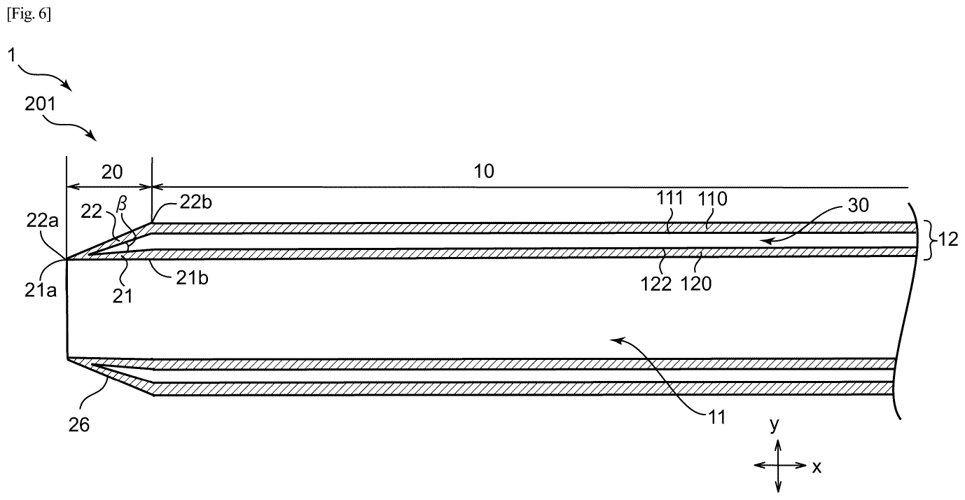

FIG. 4 is an enlarged cross-sectional view of the distal side of the catheter 1 shown in FIG. 1 and shows the non-pressurized state where pressurization by injecting a fluid into the flow path 30 is not performed. FIG. 5 is an end view, taken along the line V-V, of the catheter shown in FIG. 4. FIG. 6 is a cross-sectional view of the catheter 1 when the bag-shaped body 20 enters the pressurized state of being pressurized by injecting a fluid into the flow path 30 of the catheter 1 shown in FIG. 4.

As shown in FIGS. 4 to 6, in the catheter 1, a minimum inner diameter of the bag-shaped body 20 in the pressurized state where the bag-shaped body 20 is pressurized by injecting a fluid into the flow path 30 is larger than a minimum inner diameter of the bag-shaped body 20 in the non-pressurized state where the bag-shaped body 20 is not pressurized by injecting a fluid into the flow path 30.

In the pressurized state, the bag-shaped body 20 is preferably pressurized at a predetermined pressure. The predetermined pressure only has to have a pressure value at which the minimum inner diameter of the bag-shaped body 20 differs between before and after the pressurization. The numerical range of the pressure value is not limited. The predetermined pressure may be, for example, 0.03 MPa or higher and 4.05 MPa or lower (0.3 atm or higher and 40 atm or lower). The pressurization by injecting a fluid into the flow path 30 refers to intentional introduction of a fluid into the flow path 30 by an operator. A first predetermined pressure described later only has to be 0.03 MPa or higher and 4.05 MPa or lower, and a second predetermined pressure described later only has to be 0.03 MPa or higher and 4.05 MPa or lower.

First, the catheter 1 is delivered to a treatment site without injecting a fluid into the flow path 30. In other words, in relation to the above example, the catheter 1 is delivered to a treatment site in the state shown in FIG. 4. Thus, the bag-shaped body 20 is not pressurized by injecting a fluid into the inner space, whereby a state where the outer diameter of the portion, of the bag-shaped body 20, that has a smaller outer diameter than the outer tube 10 remains small is maintained during delivery of the catheter 1. Therefore, the catheter 1 becomes less likely to get caught on the wall of a body cavity during delivery to the treatment site, and thus the wall of the body cavity is easily prevented from being damaged by the catheter 1 during the delivery. In addition, the passability of the catheter 1 in the body cavity is also easily improved. Furthermore, after the catheter 1 reaches the treatment site, a fluid is injected into the flow path 30, whereby the bag-shaped body 20 is pressurized so that the minimum inner diameter of the bag-shaped body 20 becomes larger than that before the pressurization. In other words, in relation to the above example, the catheter 1 enters the state shown in FIG. 6. Consequently, in a case where a medical implement such as a balloon, a stent, a basket, or a needle is delivered to a treatment site through the lumens of the catheter 1, the medical implement is easily caused to project from the diameter-increased portion of the bag-shaped body 20.

Since the outer tube 10 is inserted into a body, it preferably has flexibility. Accordingly, the outer tube 10 may be deformed along a body cavity shape. The outer tube 10 preferably has elasticity for maintaining a shape thereof.

Examples of the outer tube 10 include: a hollow body formed by arranging one or a plurality of wires in a certain pattern; the hollow body of which at least one of an inner surface and an outer surface thereof is coated with a resin; a resin tube; and combination thereof (for example, those connected in a longitudinal axis direction of the outer tube 10). As the hollow body in which wires are arranged in a certain pattern, a tubular body having a mesh structure by which wires are simply crossed or knitted, or a coil in which a wire is wound is exemplified. The wire may be one or a plurality of single wires, or may be one or a plurality of stranded wires. The resin tube can be manufactured, for example, by extrusion molding. In the case where the outer tube 10 is made of a resin tube, the outer tube 10 can be composed of a single layer or a plurality of layers. A part of the outer tube 10 in the longitudinal direction x or a circumferential direction may be composed of a single layer, and another part of that may be composed of a plurality of layers.

The outer tube 10 can be composed of, for example, a synthetic resin such as a polyolefin resin (for example, polyethylene, polypropylene), a polyamide resin (for example, nylon), a polyester resin (for example, PET), an aromatic polyetherketone resin (for example, PEEK), a polyether polyamide resin, a polyurethane resin, a polyimide resin and a fluororesin (for example, PTFE, PFA, ETFE), or a metal such as stainless steel, carbon steel and nickel-titanium alloy. These may be used alone or in combination of two or more.

The outer tube 10 preferably has: an inner surface facing the lumen 11 of the outer tube 10; and an outer surface facing the outside of the outer tube 10.

The outer surface of the outer tube 10 is preferably coated with a hydrophilic polymer. The outer tube 10 can thus be easily inserted to a body cavity. Examples of the hydrophilic polymer include poly2-hydroxyethyl methacrylate, polyacrylamide, polyvinylpyrrolidone, a maleic anhydride copolymer such as a methyl vinyl ether maleic anhydride copolymer and polyethylene glycol.

The inner surface of the outer tube 10 is preferably made of a fluororesin or a polyolefin resin. Only a part of the inner surface of the outer tube 10 may be made of a fluororesin or a polyolefin resin, or the entire inner surface of the outer tube 10 may be made of a fluororesin or a polyolefin resin. This makes the outer tube 10 more easily slidable against members placed in the lumen 11, thereby improving the operability of the catheter 1.

The outer diameter of the outer tube 10 can be, for example, 1.0 mm or more, 1.1 mm or more, or 1.2 mm or more. Meanwhile, the outer diameter of the outer tube 10 can be, for example, 5.0 mm or less, 4.0 mm or less, or 3.0 mm or less, but preferably 2.0 mm or less.

The inner diameter of the outer tube 10 can be, for example, 4.0 mm or less, 3.8 mm or less, or 3.5 mm or less. Meanwhile, the inner diameter of the outer tube 10 can be, for example, 0.4 mm or more, 0.5 mm or more, or 0.6 mm or more, but preferably 0.8 mm or more or more preferably 1.2 mm or more. The inner diameter of the outer tube 10 also preferably has a value that allows an inner tube 50 having an expansion member 60 described later to pass through the outer tube 10.

As shown in FIG. 2, the outer tube 10 may have, at the distal portion of the outer tube 10, a distal opening 11a through which the lumen 11 of the outer tube 10 and the outside of the outer tube 10 are in communication with each other, and the outer tube 10 may have, at the proximal portion of the outer tube 10, a proximal opening 11b through which the lumen 11 of the outer tube 10 and the outside of the outer tube 10 are in communication with each other.

As shown in FIGS. 1 and 2, the outer tube 10 may have, at the proximal portion of the outer tube 10, a reduced diameter area 13 having a length, in the radial direction y, that is reduced toward the proximal side as seen in a direction perpendicular to the longitudinal direction x of the outer tube 10.

As shown in FIG. 2, the proximal opening 11b is preferably located in the reduced diameter area 13, and the proximal opening 11b is more preferably located in a portion tilted such that the length thereof in the radial direction y is reduced toward the proximal side as seen in a direction perpendicular to the longitudinal direction x. With this configuration, for example, a medical implement can be inserted into or removed from the outer tube 10 through the proximal opening 11b.

The reduced diameter area 13 is preferably present regardless of whether the bag-shaped body 20 is in the pressurized state or the non-pressurized state.

As shown in FIGS. 1 and 2, the catheter 1 may further have a first tubular member 40 fixed to the proximal portion of the outer tube 10. The first tubular member 40 preferably has a maximum outer diameter smaller than the maximum outer diameter of the outer tube 10 and has a lumen 41 extending in the longitudinal direction x of the outer tube 10. The first tubular member 40 preferably has a distal portion fixed to the proximal portion of the reduced diameter area 13. The lumen 41 of the first tubular member 40 and the flow path 30 are preferably in communication with each other.

The first tubular member 40 preferably has a lumen 41. The maximum outer diameter of the first tubular member 40 is preferably smaller than the maximum outer diameter of the outer tube 10. The minimum outer diameter of the first tubular member 40 may be smaller than the minimum outer diameter of the outer tube 10.

The outer diameter of the first tubular member 40 may be set to, for example, 0.60 mm or more, 0.65 mm or more, or 0.70 mm or more. Meanwhile, the outer diameter of the first tubular member 40 may be set to, for example, 3.00 mm or less, 2.95 mm or less, or 2.90 mm or less, but preferably 1.00 mm or less.

The inner diameter of the first tubular member 40 may be set to, for example, 2.50 mm or less, 2.45 mm or less, or 2.35 mm or less. Meanwhile, the inner diameter of the first tubular member 40 may be set to, for example, 0.30 mm or more, 0.35 mm or more, or 0.40 mm or more, but preferably 0.50 mm or more.

The length from the distal end of the first tubular member 40 to the proximal end of the first tubular member 40 may be set to, for example, 100 mm or more, 150 mm or more, 200 mm or more, and meanwhile, 2400 mm or less, 2350 mm or less, or 2300 mm or less.

As described above, in the mode in which the first tubular member 40 is provided, the length from the distal end 10a of the outer tube 10 to the proximal end 10b of the outer tube 10 may be set to, for example, 30 mm or more, 35 mm or more, 40 mm or more, and meanwhile, 700 mm or less, 650 mm or less, or 600 mm or less.

Regarding the configuration of the first tubular member 40, the relevant explanations of the outer tube 10 may be referred to. The material for forming the first tubular member 40 and the material for forming the outer tube 10 may be identical to or different from each other.

The first tubular member 40 preferably has an outer surface provided with a lubricating coating layer containing PTFE, PFA, or the like. Consequently, the first tubular member 40 can be easily inserted into a body cavity.

The first tubular member 40 may be provided with a position indication marker for indicating, when the catheter 1 is inserted from the distal end thereof into a body cavity, the length of the inserted portion. The position indication marker may be formed by, for example: inserting the first tubular member 40 into a lumen of a marker tube and welding the first tubular member 40 to the marker tube; or peeling a portion of the above lubricating coating layer.

The position indication marker may be provided at, for example, a position shifted from the distal end of the catheter 1 toward the proximal side by at least 600 mm, a position shifted from said distal end toward the proximal side by at least 900 mm, or a position shifted from said distal end toward the proximal side by at least 1200 mm.

The lumen 41 of the first tubular member 40 and the flow path 30 are preferably in communication with each other. Consequently, a fluid can be sent to the bag-shaped body 20 by utilizing the lumen 41 and the flow path 30. The distal portion of the first tubular member 40 and the proximal portion of the reduced diameter area 13 are preferably fixed to each other such that the lumen 41 of the first tubular member 40 and the flow path 30 are in communication with each other. The distal end of the first tubular member 40 and the proximal end of the reduced diameter area 13 are more preferably fixed to each other. It is preferable that the lumen 41 of the first tubular member 40 is not in communication with the lumen 11 of the outer tube 10. As shown in FIG. 1, the catheter 1 preferably has a hub 42 on the proximal portion thereof. The hub 42 may be connected to a proximal end portion of the first tubular member 40. The hub 42 preferably has therein a port in communication with the lumen 41 of the first tubular member 40. Consequently, a fluid can be injected into the lumen 41 through the port.

There may be a case (not shown) where the proximal end of the outer tube 10 is directly connected to the hub 42. In this case, the hub 42 preferably has therein a port in communication with the flow path 30. Consequently, a fluid can be injected into the flow path 30 through the port.

The bag-shaped body 20 may be made from a material having flexibility and/or elasticity. As the material for forming the bag-shaped body 20, any of the materials described as examples of the material for forming the outer tube 10 may be used. The material for forming the bag-shaped body 20 and the material for forming the outer tube 10 may be identical to or different from each other.

As shown in FIGS. 1 and 2, the proximal portion of the bag-shaped body 20 may be connected to the distal portion of the outer tube 10, or the proximal end of the bag-shaped body 20 may be connected to the distal end 10a of the outer tube 10.

The portion, of the bag-shaped body 20, that has a smaller outer diameter than the outer tube 10 is preferably located in the distal portion of the bag-shaped body 20, more preferably located in a distal end portion of the bag-shaped body 20, and further preferably located at the distal end of the bag-shaped body 20. Consequently, a state where an outer diameter of the bag-shaped body 20 (in particular, an outer diameter on the distal side of the bag-shaped body 20) remains small, is easily maintained. Therefore, the catheter 1 becomes less likely to get caught on the wall of a body cavity during delivery to a treatment site, and thus the wall of the body cavity is easily prevented from being damaged by the catheter 1 during the delivery. In addition, the passability of the catheter 1 in the body cavity can also be easily improved. As shown in FIG. 2, the bag-shaped body 20 may have a smaller outer diameter than the outer tube 10 over the entirety in the longitudinal direction x of the bag-shaped body 20.

The shape of the bag-shaped body 20 is not particularly limited as long as the shape is an annular shape. As shown in FIGS. 1 to 3, the bag-shaped body 20 may have, for example, a hollow frustum shape. The bag-shaped body 20 may have a hollow truncated conical shape, a hollow truncated pyramidal shape, a shape obtained by combining a hollow truncated cone and a hollow truncated pyramid (for example, a shape obtained by connecting a hollow truncated cone and a hollow truncated pyramid in the longitudinal direction x), or the like. The bag-shaped body 20 preferably has a hollow frustum shape in the non-pressurized state.

A length in the longitudinal direction x of the bag-shaped body 20 in the non-pressurized state and a length in the longitudinal direction x of the bag-shaped body 20 in the pressurized state (in a case where the catheter 1 has a bulging portion 70 described later, a state where pressurization at the second predetermined pressure or higher pressure is performed) may be equal to or different from each other.

The length of the bag-shaped body 20 in the longitudinal direction x in the non-pressurized state may be set to, for example, 1 mm or more, 3 mm or more, 5 mm or more, and meanwhile, 20 mm or less, 18 mm or less, or 15 mm or less.

The length in the longitudinal direction x of the bag-shaped body 20 in the pressurized state (in the case where the catheter 1 has the bulging portion 70, the state where pressurization at the second predetermined pressure or higher pressure is performed) may be set to, for example, 1 mm or more, 3 mm or more, or 5 mm or more, and meanwhile, 20 mm or less, 18 mm or less, or 15 mm or less.

The minimum inner diameter of the bag-shaped body 20 in the non-pressurized state preferably has a value that allows a guide wire to be inserted therein. The minimum inner diameter of the bag-shaped body 20 in the non-pressurized state may be 30% or higher, 35% or higher, or 40% or higher, and meanwhile, 70% or lower, 65% or lower, or 60% or lower of the inner diameter of the outer tube 10. The minimum inner diameter of the bag-shaped body 20 in the non-pressurized state may be set to, for example, 0.30 mm or more, 0.35 mm or more, 0.40 mm or more, and meanwhile, 2.00 mm or less, 1.95 mm or less, 1.90 mm or less.

As shown in FIG. 4, a position of the bag-shaped body 20 at which the inner diameter thereof is minimum in the non-pressurized state is preferably the distal end of the bag-shaped body 20. As shown in FIG. 6, a position of the bag-shaped body 20 at which the inner diameter thereof is minimum in the pressurized state may be the proximal end or the distal end of the bag-shaped body 20.

The minimum outer diameter of the bag-shaped body 20 in the non-pressurized state may be set to, for example, 0.31 mm or more, 0.36 mm or more, 0.41 mm or more, and meanwhile, 3.00 mm or less, 2.90 mm or less, 2.80 mm or less.

As shown in FIG. 4, a position of the bag-shaped body 20 at which the outer diameter thereof is minimum in the non-pressurized state is preferably the distal end of the bag-shaped body 20.

When a medical implement is delivered to a treatment site through the lumen 11 of the outer tube 10, the minimum inner diameter of the bag-shaped body 20 in the pressurized state of the bag-shaped body 20 preferably has a value that allows the medical implement to project from the bag-shaped body 20. The minimum outer diameter of the bag-shaped body 20 in the pressurized state may be set to, for example, 4.0 mm or less, 3.8 mm or less, 3.5 mm or less. The minimum outer diameter of the bag-shaped body 20 in the pressurized state may be set to, for example, 0.4 mm or more, 0.5 mm or more, 0.6 mm or more, but preferably 0.8 mm or more, more preferably 1.2 mm or more.

The position of the bag-shaped body 20 at which the inner diameter thereof is minimum in the pressurized state is preferably the distal end or the proximal end of the bag-shaped body 20.

It is preferable that, in the non-pressurized state, the bag-shaped body 20 does not have any outer diameter larger than the outer diameter of the outer tube 10. That is, it is preferable that, in the non-pressurized state, the bag-shaped body 20 has an outer diameter smaller than the maximum outer diameter of the outer tube 10 over the entirety of the bag-shaped body 20. By thus setting the outer diameter of the bag-shaped body 20, the outer diameter on the distal end portion side of the catheter 1 becomes small. Consequently, the catheter 1 becomes less likely to get caught on the wall of a body cavity during delivery to the treatment site, and thus the wall of the body cavity is easily prevented from being damaged by the catheter 1. In addition, the passability of the catheter 1 in the body cavity is also easily improved.

In the pressurized state, the bag-shaped body 20 may have an outer diameter equal to or larger than the outer diameter of the outer tube 10. In the pressurized state, the distal end portion of the bag-shaped body 20 may have an outer diameter smaller than the outer diameter of the outer tube 10.

It is preferable that, in the pressurized state, the bag-shaped body 20 has an outer diameter equal to or smaller than the outer diameter of the outer tube 10 as shown in FIG. 6. It is more preferable that, in the pressurized state, the bag-shaped body 20 has a maximum outer diameter equal to or smaller than the maximum outer diameter of the outer tube 10. It is further preferable that, in the pressurized state, the bag-shaped body 20 has a maximum outer diameter equal to or smaller than the minimum outer diameter of the outer tube 10.

The bag-shaped body 20 preferably has a minimum outer diameter smaller than the outer diameter of the outer tube 10. It is particularly preferable that, in the non-pressurized state, the bag-shaped body 20 has a minimum outer diameter smaller than the outer diameter of the outer tube 10 as shown in FIG. 4. Consequently, the catheter 1 becomes less likely to get caught on the wall of a body cavity during delivery to a treatment site, and thus the wall of the body cavity is easily prevented from being damaged by the catheter 1 during the delivery. In addition, the passability of the catheter 1 in the body cavity can also be easily improved. From the viewpoint of improving these advantageous effects, it is more preferable that, in the pressurized state, the bag-shaped body 20 has a maximum outer diameter equal to or smaller than the outer diameter of the outer tube 10 as shown in FIG. 4. In the pressurized state where the bag-shaped body 20 is pressurized by injecting a fluid into the flow path 30, the bag-shaped body 20 may have a minimum outer diameter smaller than the outer diameter of the outer tube 10 as shown in FIGS. 5 and 6.

As shown in FIGS. 2 to 4, in the non-pressurized state of the bag-shaped body 20, the bag-shaped body 20 may have an inner surface portion 21 facing an inner side and an outer surface portion 22 facing an outer side. In the non-pressurized state, the inner surface portion 21 may be a portion facing the lumen 25, and the outer surface portion 22 may be a portion that is located outward of the inner surface portion 21 in the radial direction and that faces the outside of the catheter 1. The distal end of the bag-shaped body 20 may serve as the boundary between the inner surface portion 21 and the outer surface portion 22. The inner space 23 is preferably present between the inner surface portion 21 and the outer surface portion 22. The inner surface portion 21 defined in the non-pressurized state may face the outside of the catheter 1 in the pressurized state. In contrast, the outer surface portion 22 defined in the non-pressurized state may face the lumen 25 in the pressurized state.

A thickness of the inner surface portion 21 in the non-pressurized state may be set to, for example, 0.005 mm or more, 0.006 mm or more, or 0.007 mm or more. Meanwhile, the thickness of the inner surface portion 21 in the non-pressurized state may be set to, for example, 0.100 mm or less, 0.090 mm or less, or 0.080 mm or less. The thickness of the inner surface portion 21 in the non-pressurized state may be equal to a thickness of the expansion member 60. The thickness of the expansion member 60 refers to, for example, the film thickness of a balloon forming the expansion member 60 or the thickness of a stent forming the expansion member 60.

A thickness of the outer surface portion 22 in the non-pressurized state may be set to, for example, 0.010 mm or more, 0.015 mm or more, or 0.020 mm or more. Meanwhile, the thickness of the outer surface portion 22 in the non-pressurized state may be set to, for example, 0.500 mm or less, 0.450 mm or less, or 0.400 mm or less. In a case where the outer tube 10 has a first outer tube 110 and a second outer tube 120 as described later, a thickness of a proximal end portion of the outer surface portion 22 in the non-pressurized state may be equal to a thickness of the first outer tube 110.

In order to set the minimum inner diameter of the bag-shaped body 20 in the pressurized state to be larger than the minimum inner diameter of the bag-shaped body 20 in the non-pressurized state, for example, it is preferable that, when a state of the bag-shaped body 20 is changed from the non-pressurized state to the pressurized state (in the case where the catheter 1 has the bulging portion 70, the state where pressurization at the second predetermined pressure or higher pressure is performed), an elongation rate (%) of a length from a distal end 21a to a proximal end 21b of the inner surface portion 21 is higher than an elongation rate (%) of a length from a distal end 22a to a proximal end 22b of the outer surface portion 22. By thus configuring the bag-shaped body 20 such that the inner surface portion 21 thereof is easily elongated to a larger extent than the outer surface portion 22 thereof, the inner diameter of the bag-shaped body 20 at the time of injecting a fluid into the flow path 30 easily becomes larger than that before the pressurization. For example, the state of the catheter 1 is easily changed from the state shown in FIG. 4 to the state shown in FIG. 6. Consequently, when a medical implement is delivered to a treatment site through the lumen 11 of the outer tube 10, the medical implement can be easily caused to project from the diameter-increased portion of the bag-shaped body 20. The elongation rate (%) of each of the portions is expressed as (the post-pressurization length of the portion)÷(the pre-pressurization length of the portion), and the same applies to cases where the elongation rate (%) is written below without any particular explanation.

The inner surface portion 21 and the outer surface portion 22 may be made from the same material or may be made from different materials.

In a case where the inner surface portion 21 and the outer surface portion 22 are made from the same material, the thickness of the inner surface portion 21 shown in FIGS. 2 and 3 is preferably smaller than a thickness of the outer surface portion 22, for example. Consequently, the inner surface portion 21 is easily elongated to a larger extent than the outer surface portion 22. Thus, when a fluid is injected into the flow path 30, the inner surface portion 21 of the bag-shaped body 20 is elongated, whereby the inner diameter of the bag-shaped body 20 easily becomes larger than that before the pressurization. For example, the state of the catheter 1 is easily changed from the state shown in FIG. 4 to the state shown in FIG. 6. Therefore, when a medical implement is delivered to a treatment site through the lumen 11, the medical implement is easily caused to project from the diameter-increased portion of the bag-shaped body 20.

In a case where the inner surface portion 21 and the outer surface portion 22 are made from different materials, an elongation rate of the material for forming the inner surface portion 21 is preferably higher than an elongation rate of the material for forming the outer surface portion 22, for example. The elongation rates mentioned herein refer to elongation rates (%) obtained as follows. That is, a first sample made from the same material as that of the inner surface portion 21 and a second sample formed in the same shape as that of the first sample and made from the same material as that of the outer surface portion 22 are prepared, and, in a state where one end and the other end of each of the samples are gripped such that the grip width is the same between the samples, the samples are pulled with a same strength (N), to obtain the elongation rates (%) of the samples. Consequently, the inner surface portion 21 of the bag-shaped body 20 is easily elongated when a fluid is injected into the flow path 30, and the inner diameter of the bag-shaped body 20 easily becomes larger than that before the pressurization. For example, the state of the catheter 1 is easily changed from the state shown in FIG. 4 to the state shown in FIG. 6. Therefore, when a medical implement is delivered to a treatment site through the lumen 11, the medical implement is easily caused to project from the diameter-increased portion of the bag-shaped body

As shown in FIG. 4, in the non-pressurized state, an angle a formed between inner walls of the bag-shaped body 20 in a cross section parallel to the longitudinal direction x of the outer tube 10 is preferably an acute angle. The angle α may be 85° or less, 80° or less, or 70° or less. Meanwhile, the angle a may be 1° or more, 3° or more, or 5° or more. With this configuration, the outer diameter of the distal end portion of the bag-shaped body 20 can be reduced. Consequently, the catheter 1 becomes less likely to get caught on the wall of a body cavity, and thus the wall of the body cavity is easily prevented from being damaged by the catheter 1. In addition, the passability of the catheter 1 in the body cavity can also be easily improved.

As shown in FIG. 6, in the pressurized state (in the case where the catheter 1 has the bulging portion 70, the state where pressurization at the second predetermined pressure or higher pressure is performed), an angle β formed between the inner walls of the bag-shaped body 20 in the cross section parallel to the longitudinal direction x of the outer tube 10 is preferably an acute angle. The angle β may be 85° or less, 80° or less, or 70° or less. Meanwhile, the angle β may be 1° or more, 3° or more, or 5° or more. With this configuration, a thickness of the distal end portion of the bag-shaped body 20 becomes small. In other words, the length between the inner surface of the inner surface portion 21 and the outer surface of the outer surface portion 22 is easily shortened. As a result, an inner diameter of the distal end portion of the bag-shaped body 20 is easily increased in the pressurized state. Thus, when a medical implement is delivered to a treatment site through the lumen 11, the medical implement is easily caused to project from the diameter-increased portion of the bag-shaped body 20.

As shown in FIG. 4, the outer tube 10 may have a wall 12 delimiting the lumen 11, and the flow path 30 may be located inside the wall 12 of the outer tube 10. With this configuration, a member for forming the flow path 30 does not need to be separately provided, whereby the catheter 1 is easily made thin.

For example, as shown in FIGS. 4 and 5, the outer tube 10 may have a first outer tube 110 and a second outer tube 120 disposed inside a lumen of the first outer tube 110, and the flow path 30 may be a space delimited by an inner surface 111 of the first outer tube 110 and an outer surface 122 of the second outer tube 120. With this configuration, a member for forming the flow path 30 does not need to be separately provided, whereby the catheter 1 is easily made thin.

Hereinafter, a thickness of each of the first outer tube 110 and the second outer tube 120 refers to the wall thickness of the tube. The thickness of the first outer tube 110 may be set to, for example, 0.010 mm or more, 0.015 mm or more, or 0.020 mm or more, and meanwhile, 0.500 mm or less, 0.450 mm or less, or 0.400 mm or less. The thickness of the second outer tube 120 may be set to, for example, 0.010 mm or more, 0.015 mm or more, or 0.020 mm or more, and meanwhile, 0.500 mm or less, 0.450 mm or less, or 0.400 mm or less.

As shown in FIG. 5, in a cross section perpendicular to the longitudinal direction x, the distance between the inner surface 111 of the first outer tube 110 and the outer surface 122 of the second outer tube 120 is preferably 0.01 mm or longer and 1.0 mm or shorter. Consequently, pressure loss at the time of sending a fluid through the flow path 30 to the distal end portion can be reduced.

The first outer tube 110 may have a distal end and a proximal end and have a lumen extending in the longitudinal direction x. The same applies to the second outer tube 120.

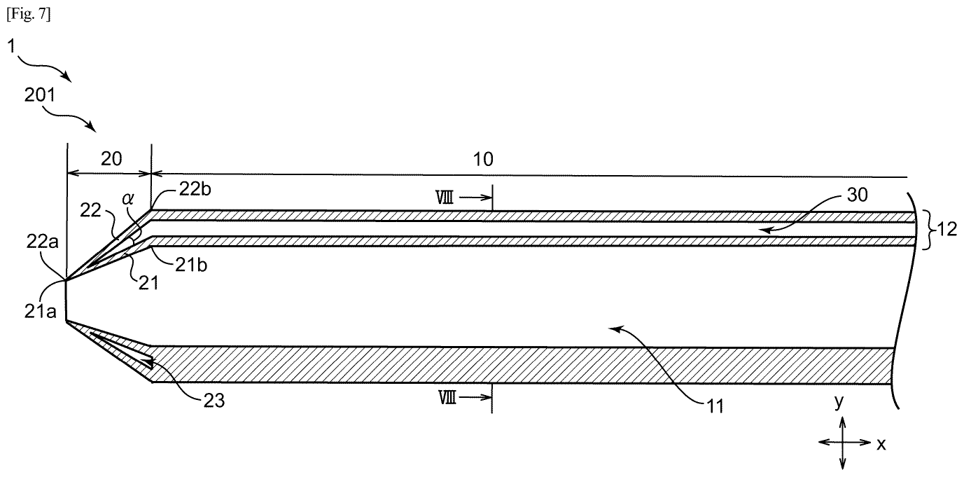

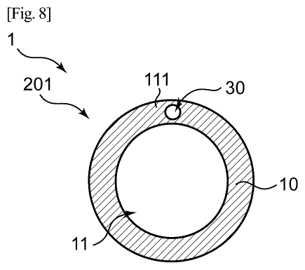

When the state of the bag-shaped body 20 is changed from the non-pressurized state to the pressurized state (in the case where the catheter 1 has the bulging portion 70, the state where pressurization at the second predetermined pressure or higher pressure is performed), the elongation rate (%) of the length from the distal end 21a to the proximal end 21b of the inner surface portion 21 is preferably higher than an elongation rate (%) of a length from the distal end 10a to the proximal end 10b of the outer tube 10. The elongation rates are particularly preferably set in this manner in a case where the flow path 30 is formed inside the wall 12 of the outer tube 10. Since the bag-shaped body 20 exhibits a higher elongation rate (%) than the outer tube 10, deformation of the outer tube 10 due to the pressure of a fluid is less likely to occur, whereby it becomes easy to stably supply, to the bag-shaped body 20, the fluid injected into the flow path 30. FIG. 7 is a cross-sectional view showing a modification of the catheter shown in FIG. 4. FIG. 8 is an end view, taken along the line VIII-VIII, of the catheter shown in FIG. 7.

As shown in FIGS. 7 and 8, the flow path 30 may extend in the longitudinal direction x inside the wall 12 of the outer tube 10. The flow path 30 may linearly extend in the longitudinal direction x. There may be a case (not shown) where the flow path extends, inside the wall 12 of the outer tube 10, in the form of a wave or helically around the lumen 11 of the outer tube 10.

As shown in FIGS. 7 and 8, in the case where the flow path 30 linearly extends in the longitudinal direction x inside the wall 12 of the outer tube 10, it is preferable that: the inner space 23 is continuously formed inside a wall of the bag-shaped body 20 over the entirety in the circumferential direction of the outer tube 10; and the inner space 23 is in communication with the linear flow path 30. Consequently, a fluid injected into the flow path 30 can be sent into the inner space 23 of the bag-shaped body 20.

Alternatively, the flow path 30 may be implemented by a member separate from the outer tube 10 instead of being formed inside the wall 12 of the outer tube 10 or between the plurality of members of composing the outer tube 10.

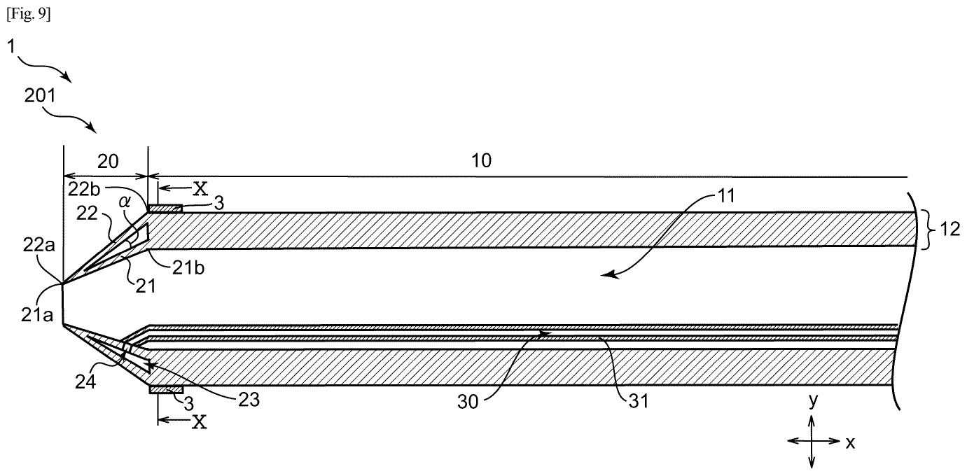

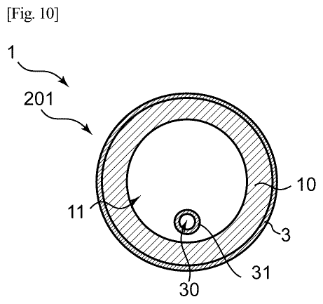

FIG. 9 is a cross-sectional view showing another modification of the catheter shown in FIG. 4. FIG. 10 is an end view, taken along the line X-X, of the catheter 1 shown in FIG. 9.

As shown in FIGS. 9 and 10, the catheter 1 may have a second tubular member 31, and the second tubular member 31 may have the flow path 30. The second tubular member is a tubular member having a lumen. Here, an example in which the second tubular member 31 is disposed inside the lumen 11 of the outer tube 10 and the lumen of the second tubular member 31 functions as the flow path 30, is described. When the second tubular member 31 is disposed inside the outer tube 10 in this manner, the catheter 1 can be easily made thin. There may be a case (not shown) where the second tubular member 31 is disposed outward of the outer tube 10 in the radial direction.

As shown in FIG. 9, in a case where the flow path 30 is formed in the second tubular member 31, it is preferable that: the bag-shaped body 20 and a distal end portion of the second tubular member 31 are fixed to each other; and the bag-shaped body 20 has a hole 24 through which the flow path 30 and the inner space 23 are in communication with each other. Consequently, a fluid injected into the flow path 30 can be sent into the inner space 23 of the bag-shaped body 20.

Regarding the configuration of the second tubular member 31, the relevant explanations of the outer tube 10 may be referred to. The material for forming the outer tube 10, the material for forming the first tubular member 40, and the material for forming the second tubular member 31 may be identical to or different from one another.

As shown in FIGS. 2 and 3, the bag-shaped body 20 preferably has the inner space 23 in communication with the flow path 30. Consequently, a fluid injected into the flow path 30 can be caused to flow into the inner space 23 formed inside the bag-shaped body 20.

As shown in FIGS. 2 and 3, the inner space 23 of the bag-shaped body 20 is preferably a space formed inside the wall of the annular bag-shaped body 20. More specifically, the inner space 23 is preferably a space present inside the wall delimiting the lumen 25 of the bag-shaped body 20. As shown in FIGS. 2 and 3, the inner space 23 is preferably present inside the wall of the bag-shaped body 20 over the entirety in circumferential direction of the wall. That is, the inner space 23 is preferably a space continuously extending in the circumferential direction inside the wall of the bag-shaped body 20. There may be a case (not shown) where the inner space 23 is present inside the wall of the bag-shaped body 20 only over a portion in the circumferential direction of the wall.

It is preferable that the inner space 23 is not in communication with the lumen 11 of the outer tube 10. It is also preferable that the inner space 23 is not in communication with the lumen 25 of the bag-shaped body 20, either. Consequently, it can be made easy to efficiently cause a fluid injected into the flow path 30 to flow into the inner space 23 formed inside the bag-shaped body 20.

As shown in FIG. 4, in the non-pressurized state, a length in the radial direction y of the inner space 23 in a cross section passing through a central axis of the outer tube 10 and extending along the longitudinal direction x is preferably reduced toward the distal end from the proximal end of the inner space 23. Consequently, the outer diameter of the distal end portion of the bag-shaped body 20 is easily made small, whereby the wall of the body cavity is easily prevented from being damaged by the catheter 1 during delivery to the treatment site. In addition, the passability of the catheter 1 in the body cavity can also be easily improved.

As shown in FIG. 6, in the pressurized state (in the case where the catheter 1 has the bulging portion 70, the state where pressurization at the second predetermined pressure or higher pressure is performed), the length in the radial direction y of the inner space 23 in the cross section passing through the central axis of the outer tube 10 and extending along the longitudinal direction x is preferably reduced toward the distal end from the proximal end of the inner space 23. Consequently, the outer diameter of the distal end portion of the bag-shaped body 20 in the pressurized state is easily made small, whereby the bag-shaped body 20 is easily inhibited from being unintentionally brought into contact with the wall of a body cavity.

As shown in FIG. 4, in the non-pressurized state, the length in the radial direction y of the inner space 23 in the cross section passing through the central axis of the outer tube 10 and extending along the longitudinal direction x is preferably equal to or smaller than a length in the radial direction y of the flow path 30 in said cross section. Consequently, the outer diameter of the distal end portion of the bag-shaped body 20 is easily made small, whereby the wall of the body cavity is easily prevented from being damaged by the catheter 1 during delivery to the treatment site. In addition, the passability of the catheter 1 in the body cavity can also be easily improved.

As shown in FIG. 6, in the pressurized state (in the case where the catheter 1 has the bulging portion 70, the state where pressurization at the second predetermined pressure or higher pressure is performed), the length in the radial direction y of the inner space 23 in the cross section passing through the central axis of the outer tube 10 and extending along the longitudinal direction x is preferably equal to or smaller than the length in the radial direction y of the flow path 30 in said cross section. Consequently, the outer diameter of the bag-shaped body 20 in the pressurized state is easily made small, whereby the bag-shaped body 20 is easily inhibited from being unintentionally brought into contact with the wall of a body cavity.

As shown in FIGS. 1, 2, 4, and 6, it is preferable that the bag-shaped body 20 is disposed on the distal side relative to the distal end 10a of the outer tube 10 and has a tapered portion 26 having an outer diameter that is reduced toward the distal side. The bag-shaped body 20 may have the tapered portion merely at a portion thereof, but it is more preferable that the bag-shaped body 20 is, over the entirety thereof (i.e., from the distal end to the proximal end of the bag-shaped body 20), formed as the tapered portion.

As shown in FIG. 4, it is preferable that the bag-shaped body 20 in the non-pressurized state has the tapered portion 26. As shown in FIG. 4, it is more preferable that the bag-shaped body 20 in the non-pressurized state is, over the entirety thereof, formed as the tapered portion 26. Consequently, the wall of the body cavity is easily prevented from being damaged by the catheter 1 during delivery to the treatment site. In addition, the passability of the catheter 1 in the body cavity can also be easily improved.

As shown in FIG. 6, the bag-shaped body 20 in the pressurized state may have the tapered portion 26. As shown in FIG. 6, the bag-shaped body 20 in the pressurized state may be, over the entirety thereof, formed as the tapered portion 26.

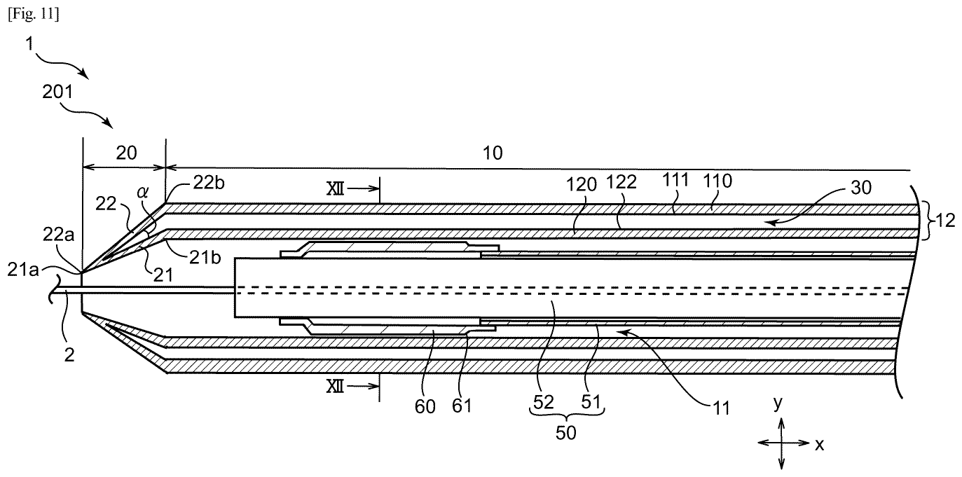

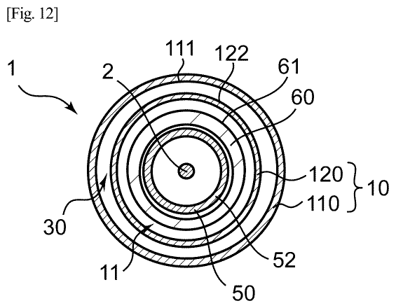

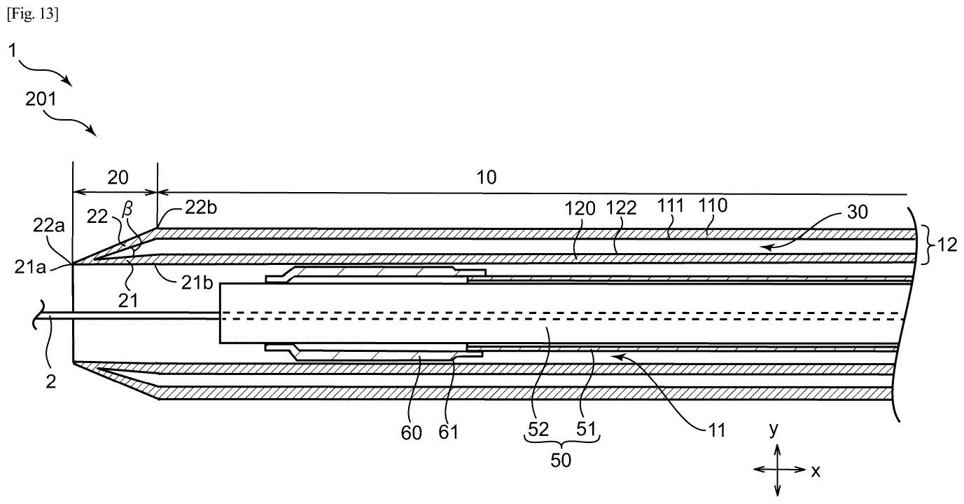

FIG. 11 is a cross-sectional view showing another modification of the catheter shown in FIG. 4. Meanwhile, regarding a guide wire 2 and a second inner tube 52 described later, FIG. 11 is a side view thereof. FIG. 12 is an end view, taken along the line XII-XII, of the catheter shown in FIG. 11. FIG. 13 is a cross-sectional view (partial side view) of the catheter shown in FIG. 11 in the pressurized state. In FIGS. 11 and 13, a portion, of the guide wire 2, that is disposed inside a lumen of the inner tube 50 is shown as broken lines.

As shown in FIGS. 11 to 13, the catheter 1 may have, inside the lumen 11 of the outer tube 10, the inner tube 50 disposed in a state of being movable relative to the outer tube 10.

The inner tube 50 may have a lumen extending in the longitudinal direction x. As shown in FIGS. 11 to 13, the lumen of the inner tube 50 can be used as an insertion path for the guide wire 2 or the like.

The inner tube 50 may be composed of one or a plurality of tubes. As shown in FIGS. 11 and 13, the inner tube 50 may have: a first inner tube 51 disposed inside the lumen 11 of the outer tube 10; and the second inner tube 52 disposed inside a lumen of the first inner tube 51.

Regarding the configuration of the inner tube 50, the relevant explanations of the outer tube 10 may be referred to. The material for forming each of the outer tube 10, the first tubular member 40, and the second tubular member 31, and the material for forming the inner tube 50, may be identical to or different from each other. The materials forming the first inner tube 51 and the second inner tube 52 may be identical to or different from each other.

The second inner tube 52 is preferably such that an inner surface of the second inner tube 52 has a portion made from a fluorine-based resin or a polyolefin-based resin. Merely a portion of the inner surface of the second inner tube 52 may be made from a fluorine-based resin or a polyolefin-based resin, or the entirety of the inner surface of the second inner tube 52 may be made from a fluorine-based resin or a polyolefin-based resin. Consequently, the second inner tube 52 easily slips on the guide wire 2, whereby the operability of the catheter 1 can be improved.

The catheter 1 may further have a control mechanism which restricts movement of the inner tube 50 in the longitudinal direction x inside the lumen 11 of the outer tube 10. The control mechanism enables the catheter 1 to be delivered to a treatment site in a state where movement of the inner tube 50 in the longitudinal direction x relative to the outer tube 10 is restricted, whereby the catheter 1 can be easily delivered to the treatment site.

Examples of the mode of the control mechanism include a mode (not shown) in which the first tubular member 40 is provided with a gripping member capable of gripping the inner tube 50. By gripping the inner tube 50 by the gripping member, movement of the inner tube 50 in the longitudinal direction x relative to the outer tube 10 can be temporarily restricted. Meanwhile, by detaching the inner tube 50 from the gripping member, the state of the inner tube 50 can be returned to the state of being movable relative to the outer tube 10. Examples of the gripping member include: a rubber member provided with a slit for sandwiching and gripping the inner tube 50; and a clip made from a rigid resin.

As shown in FIGS. 11 to 13, the catheter 1 preferably includes the expansion member 60 which is disposed on the distal portion of the inner tube 50 and which is expanded in the radial direction y.

As the expansion member 60, for example, a balloon or a stent may be used.

The balloon is preferably made from a resin. Examples of the resin for forming the balloon include polyamide-based resins, polyester-based resins, polyurethane-based resins, polyolefin-based resins, vinyl chloride-based resins, silicone-based resins, and natural rubbers. These types of resins may be used singly, or two or more of these types of resins may be used in combination. Among these resins, a polyamide-based resin, a polyester-based resin, or a polyurethane-based resin is suitably used. From the viewpoint of thinning the balloon and the flexibility of the balloon, an elastomer resin may be used.

In a case where the inner tube 50 has the first inner tube 51 and the second inner tube 52 and the expansion member 60 is a balloon, it is preferable that, as shown in FIGS. 11 and 13, the expansion member 60 has a distal end portion fixed to the distal portion of the second inner tube 52 and has a proximal end portion fixed to the distal portion of the first inner tube 51. The expansion member 60 can be expanded by causing a fluid to flow into the lumen of the first inner tube 51.

The stent is, for example, a structure that allows the diameter thereof to be increased and that is formed in a network pattern such as a mesh pattern. The stent may have a tubular shape. The stent is stretched/contracted in, for example, the circumferential direction and the axial direction. The stent may include a plurality of struts. The stent can be formed in a pattern of mutually connected structural elements. Examples of the type of the stent include: a type in which the stent is in the form of a coil made from one linear metal or a macromolecular material; a type in which the stent has been obtained by performing, with a laser or the like, cut-out machining on a metal tube or a tube made from a macromolecular material; a type in which the stent has been assembled by welding linear portions; and a type in which the stent has been made by weaving a plurality of linear metals.

The stent is preferably made from a shape-memory alloy or a shape-memory resin. The stent may be made from, for example, stainless steel such as SUS304 or SUS316, platinum, nickel, cobalt, chromium, titanium, tungsten, aluminum, gold, silver, an Ni—Ti alloy, a Co—Cr alloy, or the like.

The stent may be a self-expandable stent or a balloon-expandable stent.

By injecting a fluid into the flow path 30 after the catheter 1 reaches a treatment site, the bag-shaped body 20 is pressurized, whereby the minimum inner diameter of the bag-shaped body 20 becomes larger than that before the pressurization. For example, the state of the catheter 1 is changed from the state shown in FIG. 11 to the state shown in FIG. 13 by injecting a fluid. The catheter 1 is delivered to a treatment site in a state where the expansion member 60 is disposed inside the lumen 11 of the outer tube 10 as shown in FIG. 11, and then the minimum inner diameter of the bag-shaped body 20 is increased as shown in FIG. 13, whereby the inner tube 50 provided with the expansion member 60 is easily caused to project from the diameter-increased portion.

As the expansion member 60, a balloon or a stent having a coating layer on an outer surface 61 thereof may be used. The catheter 1 is delivered to a treatment site in a state where the balloon or the stent is disposed inside the lumen 11 of the outer tube 10 as shown in FIG. 11, and then the minimum inner diameter of the bag-shaped body 20 is increased as shown in FIG. 13, whereby the inner tube 50 provided with the balloon or the stent can be caused to project from the diameter-increased portion. Therefore, the coating layer of the balloon or the stent can be inhibited from peeling at the time of delivering the balloon or the stent to the treatment site.

The coating layer can be formed by applying a coating agent onto the outer surface of the balloon or the stent. The coating layer may be formed merely on a portion of the outer surface of the balloon or the stent or may be formed on the entire outer surface of the balloon or the stent.

The coating layer may contain a lubricating coating agent or a bioactive drug.

Examples of the lubricating coating agent include: silicone-based coating agents such as silicone and polydimethylsiloxane; acrylic-based coating agents such as sodium (meth)acrylate, butyl (meth)acrylate, methyl (meth)acrylate, ethyl (meth)acrylate, propyl (meth)acrylate, octyl (meth)acrylate, and 2,2,2-trifluoroethyl methacrylate; fluorine-based coating agents such as polytetrafluoroethylene; and hydrophilic coating agents such as polyvinylpyrrolidone, hyaluronic acid, and polyethylene glycol.

Examples of the bioactive drug include paclitaxel, docetaxel, sirolimus, temsirolimus, everolimus, zotarolimus, biolimus A9, cilostazol, cyclosporine, and NF-KB Decoy Oligo. These types of bioactive drugs may be used singly, or two or more of these types of bioactive drugs may be used in combination. The balloon or the stent may be coated with the bioactive drug alone or may be coated with a mixture obtained by adding, as appropriate, an additive to the bioactive drug.

The length of the catheter 1 from the distal end to the proximal end may be set to, for example, 200 mm or more, 250 mm or more, or 300 mm or more. Meanwhile, the length of the catheter 1 from the distal end to the proximal end can be, for example, 2500 mm or less, 2450 mm or less, or 2400 mm or less.

A proximal end portion of the catheter 1 is provided with, for example, the hub 42, a controller (not shown) to be used for operating the catheter 1, and another device (not shown), and at least one of these members may further have a mechanism capable of fixation of the relative position between the members such that this member does not move relative to at least one of the other members. The mechanism is preferably capable of fixation so as not to shift the relative position between at least two members among the hub 42, the controller, and the other device. Here, the phrase “capable of fixation” means not only being capable of obtaining a fixed state so as not to shift the relative position between the at least two members, but also being capable of releasing the at least two members from the fixed state such that one of the members is movable relative to the other member(s).

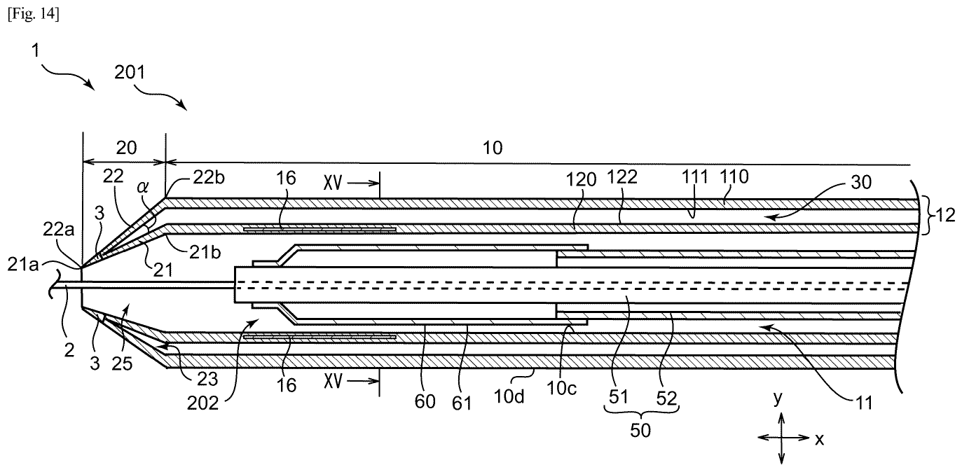

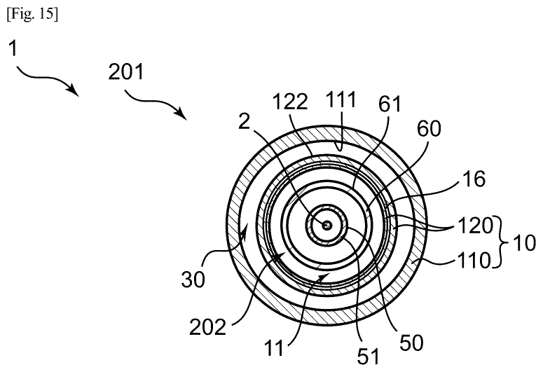

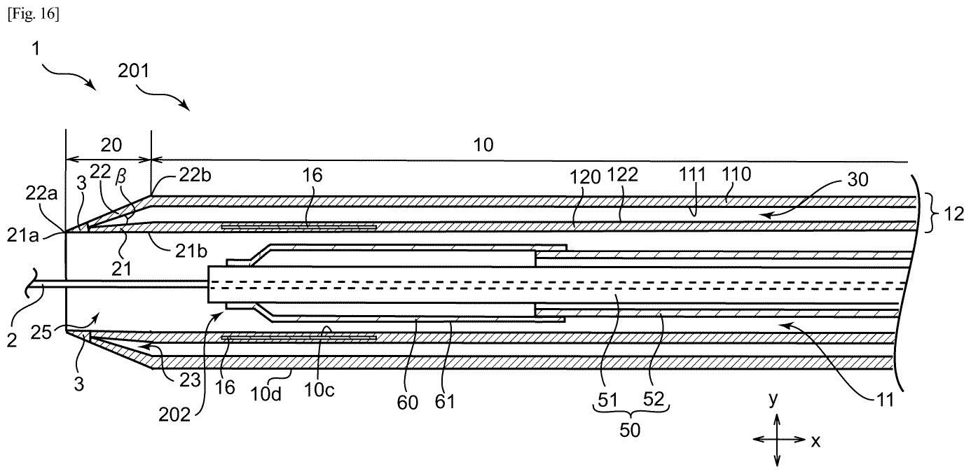

FIG. 14 is a cross-sectional view (partial side view) showing a modification of the catheter 1 shown in FIG. 4. FIG. 15 is an end view, taken along the line XV-XV, of the catheter 1 shown in FIG. 14. FIG. 16 is a cross-sectional view (partial side view) when the catheter 1 shown in FIG. 14 enters the pressurized state of being pressurized by injecting a fluid into the flow path.

As shown in FIGS. 14 to 16, a reinforcing member 16 may be disposed between the flow path 30 and the lumen 11 of the outer tube 10. Consequently, deformation of the flow path 30 is easily suppressed when a diameter-increasing member is disposed inside the lumens of the catheter 1.

The reinforcing member 16 may be made from a metal or a resin. As the metal or the resin for forming the reinforcing member 16, any of the materials described as examples of the material for forming the outer tube 10 may be used.

The reinforcing member 16 is preferably implemented by a tube, a braid obtained by braiding a wire material, a coil obtained by winding a wire material, or a combination thereof. The wire material may be a single wire or a stranded wire.

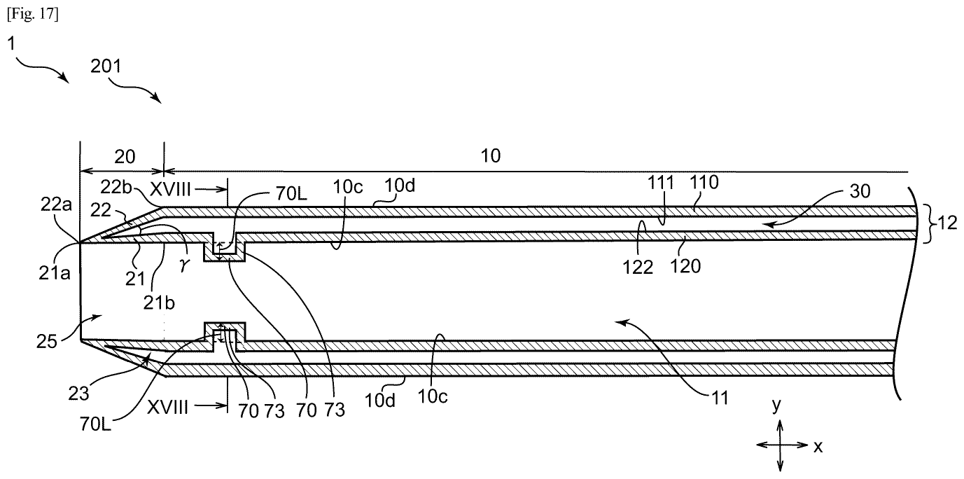

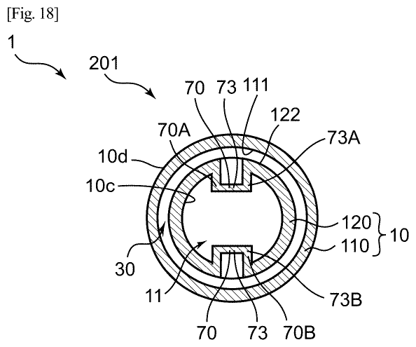

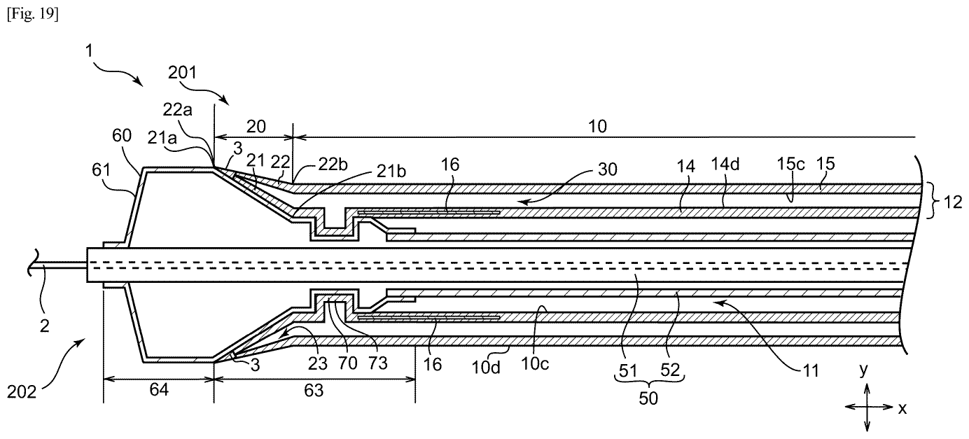

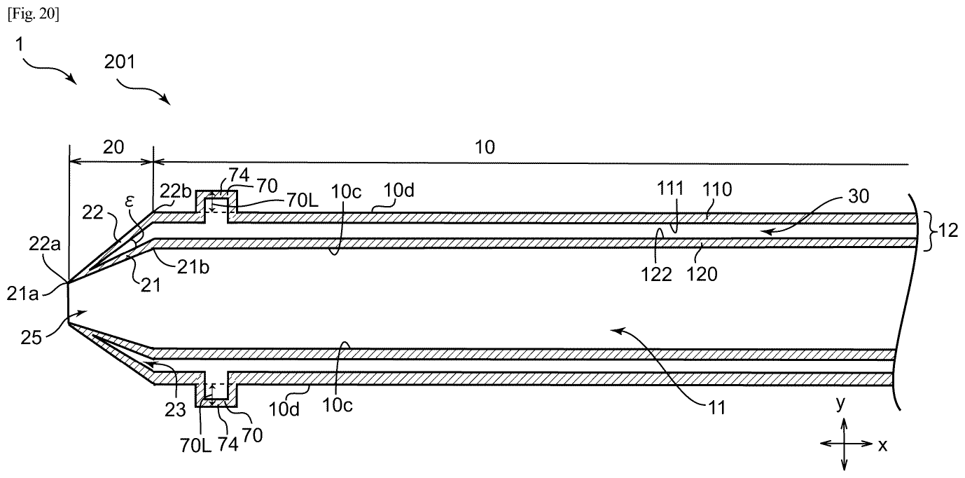

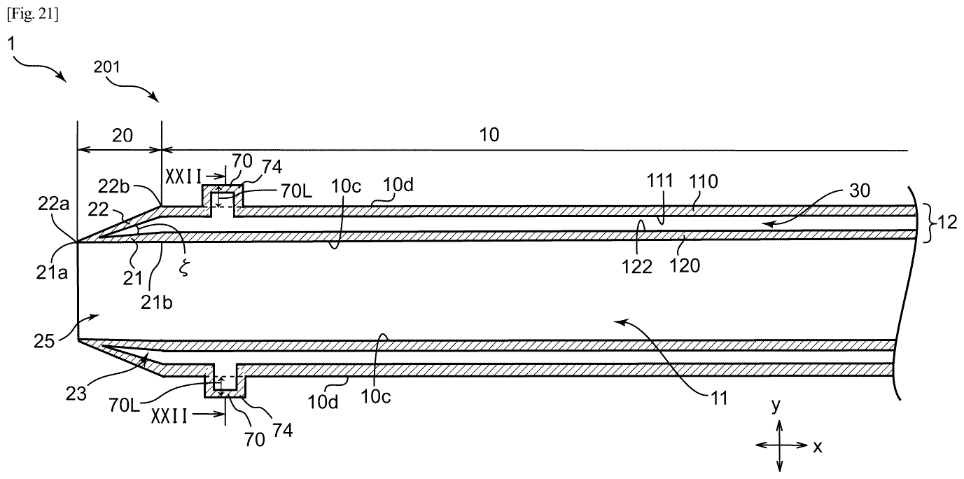

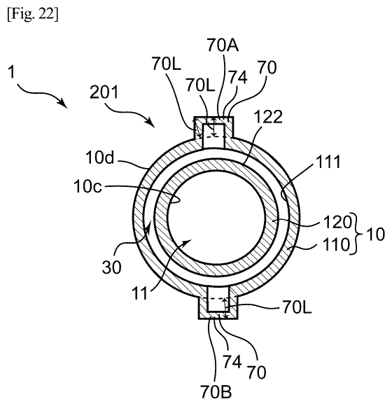

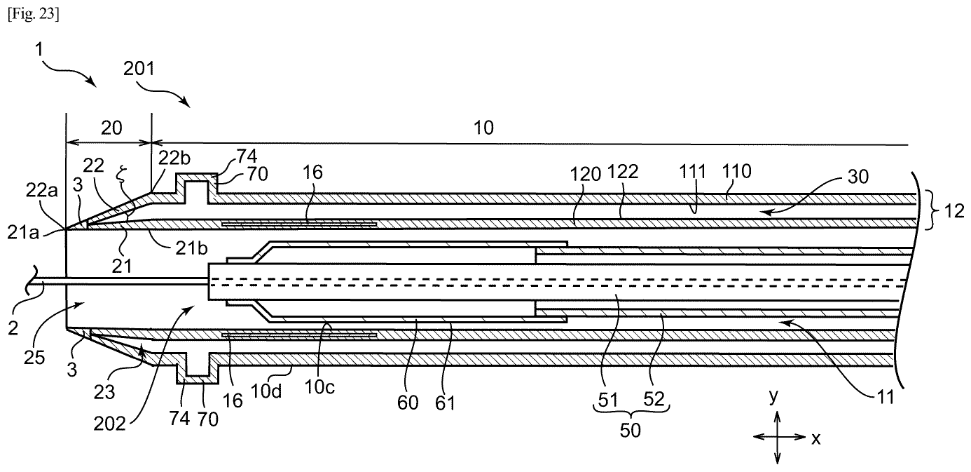

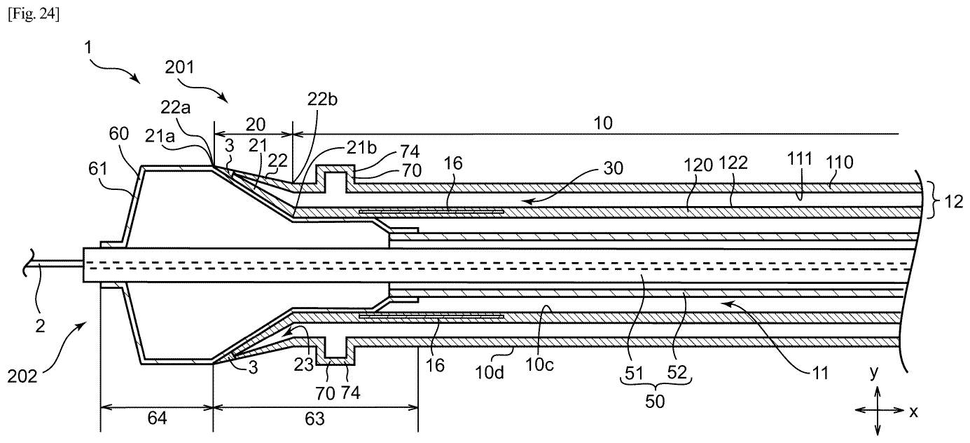

FIG. 17 is a cross-sectional view of an example of a catheter according to another embodiment of the present invention and shows a pressurized state where the catheter is pressurized by injecting a fluid into the flow path. FIG. 18 is a cross-sectional view, taken along the line XVIII-XVIII, of the catheter shown in FIG. 17. FIG. 19 is a cross-sectional view (partial side view) of a modification of the catheter shown in FIG. 16 and shows a pressurized state where the catheter is pressurized by injecting a fluid into the flow path. FIG. 20 is a cross-sectional view of an example of a catheter according to still another embodiment of the present invention and shows a state where a bulging portion is pressurized at the first predetermined pressure or higher pressure by injecting a fluid into the flow path. FIG. 21 is a cross-sectional view showing a state where the bag-shaped body of the catheter shown in FIG. 20 is pressurized at the second predetermined pressure or higher pressure by injecting a fluid into the flow path. FIG. 22 is a cross-sectional view, taken along the line XXII-XXII, of the catheter shown in FIG. 21. FIG. 23 is a cross-sectional view of an example of a catheter according to still another embodiment of the present invention and shows a state where the bag-shaped body is pressurized by injecting a fluid into the flow path. FIG. 24 is a cross-sectional view (partial side view) of a modification of the catheter shown in FIG. 23 and shows a pressurized state where the catheter is pressurized by injecting a fluid into the flow path.

As shown in FIGS. 17 to 24, the catheter 1 may further include at least one bulging portion 70 which bulges inward in the radial direction y of the outer tube 10 or outward in the radial direction y of the outer tube 10. The at least one bulging portion 70 is preferably provided to the outer tube 10 or the bag-shaped body 20 and more preferably provided to a surface of the outer tube 10. In FIGS. 17 to 19, at least one bulging portion 70 which bulges inward in the radial direction y (hereinafter, sometimes referred to as “inward bulging portion 73”) is provided. Meanwhile, in FIGS. 20 to 24, at least one bulging portion 70 which bulges outward in the radial direction y (hereinafter, sometimes referred to as “outward bulging portion 74”) is provided.

The bulging portion 70 preferably has an inner space into which a fluid for inflating the bulging portion 70 can be introduced. The bulging portion 70 is preferably inflated when the bulging portion is pressurized by injecting a fluid into the flow path 30 (a first flow path 30) and/or a second flow path described later. The above flow path 30 may function not only as the path for supplying a fluid for pressurizing the bag-shaped body 20 but also as a path for supplying a fluid for inflating the bulging portion 70. In this case, the flow path 30 is preferably in communication with the inner space of the bag-shaped body 20 and in communication with the inner space of the bulging portion 70.

In a state where, as shown in FIG. 4, a fluid is not injected into the inner space of the bulging portion 70 and the bulging portion 70 is not pressurized, a length by which the bulging portion 70 projects inward or outward only has to be smaller than that in a state where the bulging portion 70 is pressurized, and the bulging portion 70 does not have to be visually recognizable.

As a material for forming the bulging portion 70, any of the materials described as examples of the material for forming the outer tube 10, any of materials described as examples of the material for forming a balloon described later, or the like may be used. The material for forming the bulging portion 70 and the material for forming the outer tube 10 may be identical to or different from each other.

As shown in FIGS. 17 and 20, the at least one bulging portion 70 is preferably inflated so as to bulge inward or outward in the radial direction y of the outer tube 10 when the at least one bulging portion 70 enters a state of being pressurized at the first predetermined pressure or higher pressure by injecting a fluid into the flow path 30. Consequently, the bulging portion 70 bulges when the bulging portion 70 enters a state of being pressurized at the first predetermined pressure or higher pressure. As shown in FIGS. 6 and 21, a minimum inner diameter of the bag-shaped body 20 in a state where the bag-shaped body 20 is pressurized at the second predetermined pressure or higher pressure by injecting a fluid into the flow path 30 is preferably larger than the minimum inner diameter of the bag-shaped body 20 in the non-pressurized state where pressurization by injecting a fluid into the flow path 30 is not performed. Consequently, the minimum inner diameter of the bag-shaped body 20 is increased when the bag-shaped body 20 enters a state of being pressurized at the second predetermined pressure or higher pressure.

The first predetermined pressure is the lower limit value of the pressure at which the bulging portion 70 is inflated, and the second predetermined pressure is the lower limit value of the pressure at which the bag-shaped body 20 is inflated. The first predetermined pressure and the second predetermined pressure may have values equal to or different from each other.

The diameter of the bag-shaped body 20 may be increased after the bulging portion 70 is caused to bulge. The bulging portion 70 may be caused to bulge after the diameter of the bag-shaped body 20 is increased. Increase of the diameter of the bag-shaped body 20 and bulging of the bulging portion 70 may occur simultaneously.

The outer tube 10 may have the bulging portion 70. For example, as shown in FIGS. 17 to 24, the bulging portion 70 may be a portion that projects inward or outward in the radial direction y of the outer tube 10 as a result of inflating of a portion of the outer tube 10. The bulging portion 70 may have a configuration (not shown) different from that of the outer tube 10. For example, the bulging portion 70 may be a bag-shaped member disposed inside the lumen 11 of the outer tube 10. In this case, a configuration may be employed in which a fluid is injected through a tubular member connected to the bag-shaped member. In this case, a lumen of the tubular member functions as the flow path 30.

In a case where a portion of the outer tube 10 is formed as the bulging portion 70 as shown in FIGS. 17 to 24, a wall thickness of a first region, of the outer tube 10, that is formed as the bulging portion 70 is preferably smaller than a wall thickness of a second region, of the outer tube 10, that is formed as the portion other than the bulging portion 70. Consequently, the first region is easily elongated to a larger extent than the second region, whereby the first region is easily caused to bulge.

In the case where a portion of the outer tube 10 is formed as the bulging portion 70 as shown in FIGS. 17 to 24, an elongation rate of a material for forming the first region is preferably higher than an elongation rate of a material for forming the second region. Consequently, the first region is easily elongated to a larger extent than the second region, whereby the first region is easily caused to bulge. The elongation rates mentioned herein refer to elongation rates (%) obtained as follows. That is, a third sample made from the same material as that of the first region and a fourth sample formed in the same shape as that of the third sample and made from the same material as that of the second region are prepared, and, in a state where a first end and a second end of each of the third and fourth samples are gripped such that the grip width is the same length between the samples, the samples are pulled with a same strength (N), to obtain the elongation rates (%) of the samples.

When the diameter of the expansion member 60 has been increased in a state where the expansion member 60 is disposed inside the lumen 25 of the bag-shaped body 20 as shown in FIGS. 19 and 24, the outer diameter of the bag-shaped body 20 may be reduced toward the proximal side. In this case, the outer diameter may be reduced toward the proximal side over the entirety of the bag-shaped body 20.

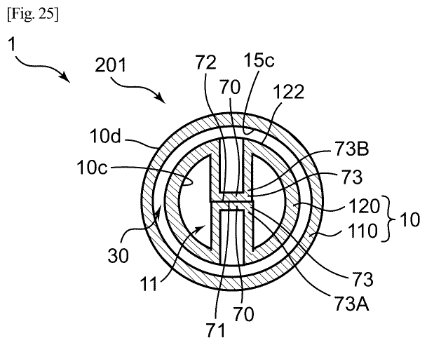

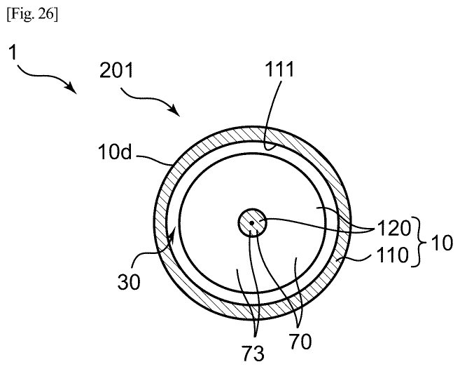

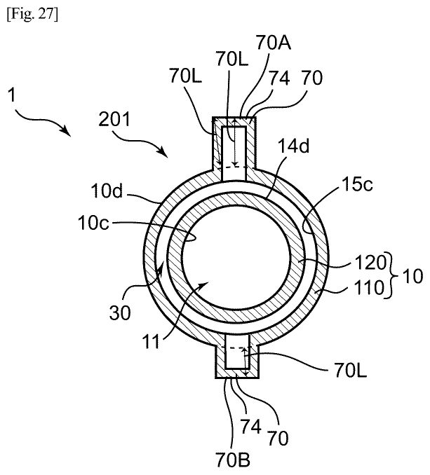

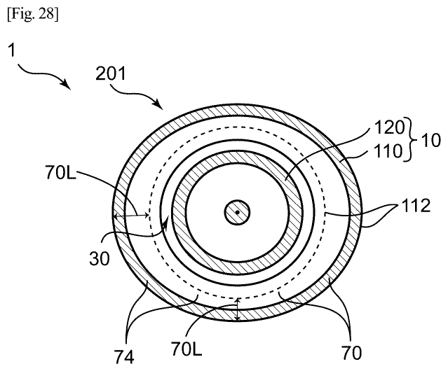

FIGS. 25 and 26 are each a cross-sectional view showing a modification of the catheter 1 shown in FIG. 18. FIGS. 27 and 28 are each a cross-sectional view showing a modification of the catheter 1 shown in FIG. 22.

The at least one bulging portion 70 may be present over a portion in the circumferential direction of the outer tube 10 as shown in FIGS. 18, 22, 25, and 27 or may be present over the entirety in the circumferential direction of the outer tube 10 as shown in FIGS. 26 and 28.

The outer tube 10 may have one bulging portion 70 alone or may have a plurality of the bulging portions 70.

In a case where the at least one bulging portion 70 has a first bulging portion 70A and a second bulging portion 70B arranged at positions that mutually differ in the circumferential direction as shown in FIGS. 18 and 22, a bulging height of the first bulging portion 70A and a bulging height of the second bulging portion 70B may be equal to each other. The bulging height of the bulging portion 70 refers to the length of the portion bulging in the radial direction y.

As shown in FIG. 27, the at least one bulging portion 70 may have a first bulging portion 70A and a second bulging portion 70B arranged at positions that mutually differ in the circumferential direction, and the bulging height of the first bulging portion 70A and the bulging height of the second bulging portion 70B may differ from each other. For example, the bulging height of the first bulging portion 70A may be higher than the bulging height of the second bulging portion 70B. As shown in FIG. 26, the bulging height of the at least one bulging portion 70 in the radial direction y may be unchanged, i.e., may have a fixed value, in the circumferential direction of the outer tube 10.

As shown in FIG. 28, the bulging height of the at least one bulging portion 70 in the radial direction y may vary in the circumferential direction of the outer tube 10. For example, the bulging height of the at least one bulging portion 70 in the radial direction y may vary according to a position of the outer tube 10 in the circumferential direction. The bulging height of one bulging portion 70 in the radial direction y may vary according to the position of the outer tube 10 in the circumferential direction.

As shown in FIGS. 17 and 20, the at least one bulging portion 70 is preferably located on the proximal side relative to the proximal end of the bag-shaped body 20. The at least one bulging portion 70 may be located on the proximal side relative to the distal end 10a of the outer tube 10. The at least one bulging portion 70 is preferably provided to the distal portion of the outer tube 10.

A frictional force of a surface of the bulging portion 70 is preferably higher than a frictional force of a portion, of the surface of the outer tube 10, other than the surface of the bulging portion 70. Consequently, when the bulging portion 70 is brought into contact with another member of the catheter 1 or the wall of a body cavity, movement of the catheter 1 or the member of the catheter 1 in the longitudinal direction x is easily restricted. The frictional force of each of the surfaces can be adjusted by, for example, changing the coating, the surface roughness, or the material of the surface.

When the diameter of the expansion member 60 has been increased in the state where the expansion member 60 is disposed inside the lumen 25 of the bag-shaped body 20 as shown in FIGS. 19 and 24, the minimum outer diameter of the bag-shaped body 20 may be equal to or larger than the outer diameter of the outer tube 10.

The flow path 30 may allow a fluid to be injected therethrough to the bulging portion 70. Consequently, a fluid for pressurizing the bag-shaped body 20 can also serve as a fluid for pressurizing the bulging portion 70. The catheter 1 may have, in addition to the flow path 30, a second flow path (not shown) through which a fluid is supplied into the inner space of the bulging portion 70. The second flow path may have one end and another end in the extension direction thereof.

The second flow path may be provided independently of the flow path 30. The second flow path is connected to the bulging portion 70 but does not have to be connected to the bag-shaped body 20. Adjustment as to which out of the timing of causing the bulging portion 70 to bulge and the timing of increasing the diameter of the bag-shaped body 20 is to precede the other timing, can be performed by adjusting the order of injecting the fluids. For example, the catheter 1 may have a second fluid supply unit connected to the one end of the second flow path, and the other end of the second flow path may be connected to the bulging portion 70.

The second flow path may be connected to the flow path 30. The second flow path may be in communication with the flow path 30. That is, the path for supplying the fluid for pressurizing the bulging portion 70 may share a portion of the path for supplying the fluid for pressurizing the bag-shaped body 20. Consequently, the fluid for pressurizing the bag-shaped body 20 can also serve as the fluid for pressurizing the bulging portion 70. For example, the catheter 1 may have a second fluid supply unit connected to the one end of the second flow path, and the other end of the second flow path may be connected to an intermediate portion of the flow path 30.

A fluid injected toward the bag-shaped body 20 may pass through a flow path (for example, flow path 30 or second flow path) which allows the fluid to be injected therethrough to the bulging portion 70. A fluid injected toward the bulging portion 70 may pass through the flow path 30 which allows the fluid to be injected therethrough to the bag-shaped body 20.

The type of the fluid to be caused to flow into the flow path 30 and/or the second flow path is not particularly limited. For example, a liquid such as a physiological saline, a contrast medium, or a liquid mixture thereof, or a gas such as air, nitrogen gas, or carbon dioxide gas, may be used. The fluids to be caused to flow into the flow path 30 and the second flow path may be of the same type or different types.

As shown in FIGS. 17 to 19, the inward bulging portion 73 is inflated so as to project inward as a result of being pressurized by injecting a fluid into the inner space of the inward bulging portion 73. At this time, the inward bulging portion 73 easily comes into contact with a member (for example, inner tube 50) disposed inside the lumen 11 of the outer tube 10, and thus can restrict movement of the inner tube 50 relative to the outer tube 10.

The catheter 1 provided with the inward bulging portion 73 can be used according to, for example, the following procedure. First, the catheter 1 which is in the non-pressurized state as shown in FIGS. 4 and 14 is delivered to a treatment site. After the delivery to the treatment site, a fluid is injected into the flow path 30 so as to pressurize the bag-shaped body 20 as shown in FIGS. 6 and 16. After the bag-shaped body 20 is pressurized, the inner tube 50 is caused to project from the outer tube 10. Thereafter, the fluid is further injected through the flow path 30 so as to pressurize the inward bulging portion 73 and cause the inward bulging portion 73 to project inward as shown in FIGS. 17 to 19. Consequently, as shown in FIG. 19, the inward bulging portion 73 comes into contact with the inner tube 50, whereby movement, in the longitudinal direction, of the inner tube 50 projecting from the outer tube 10 can be restricted.

In a case where a flow path for injecting a fluid into the inward bulging portion 73 and a flow path for injecting a fluid into the bag-shaped body 20 are in communication with each other, or in a case where the flow path for injecting a fluid into the inward bulging portion 73 and the flow path for injecting a fluid into the bag-shaped body 20 have a portion shared by each other, the second predetermined pressure is preferably lower than the first predetermined pressure. Consequently, the inward bulging portion 73 can be caused to bulge after the diameter of the bag-shaped body 20 is increased. More specifically, the catheter 1 shown in FIGS. 4 and 14 is such that: the bag-shaped body 20 is pressurized at the second predetermined pressure or higher pressure so that the minimum inner diameter of the bag-shaped body 20 can be increased as shown in FIGS. 6 and 16; and the inward bulging portion 73 is pressurized at the first predetermined pressure (higher than the second predetermined pressure) or higher pressure so that the inward bulging portion 73 can be caused to bulge as shown in FIGS. 17 to 19.

As shown in FIG. 25, the at least one bulging portion 70 may have a first inward bulging portion 73A and a second inward bulging portion 73B which bulge inward in the radial direction y of the outer tube 10 and which are arranged at positions that mutually differ in the circumferential direction. In the pressurized state, the first inward bulging portion 73A preferably comes into contact with the second inward bulging portion 73B. Consequently, a member disposed inside the lumen 11 can be sandwiched between the first inward bulging portion 73A and the second inward bulging portion 73B. For example, in a case where the inner tube 50 and the guide wire 2 are inserted into the lumen 11 as shown in FIG. 14, the guide wire 2 can be sandwiched by causing the two inward bulging portions 73A and 73B to bulge after: the distal end of the guide wire 2 is located on the distal side relative to the proximal ends of the two inward bulging portions 73A and 73B; and the inner tube 50 is located on the proximal side relative to the proximal ends of the two inward bulging portions 73A and 73B. Consequently, movement of the guide wire 2 in the longitudinal direction x is easily restricted. Therefore, the inner tube 50 is easily removed without removing the guide wire 2 from the lumen 11.

As shown in FIG. 25, in a case where the first inward bulging portion 73A and the second inward bulging portion 73B are each formed of a portion of the inner wall of the outer tube 10, the first inward bulging portion 73A may have a first surface portion 71 which is located on an inner side in the radial direction y of the outer tube 10 and which is a portion of the surface of the first inward bulging portion 73A, and the second inward bulging portion 73B may have a second surface portion 72 which is located on an inner side in the radial direction y of the outer tube 10 and which is a portion of the surface of the second inward bulging portion 73B, the second surface portion 72 being a portion different from the first surface portion 71. The first surface portion 71 and the second surface portion 72 may each be rephrased as a surface facing the lumen 11. The guide wire 2 can be sandwiched between the first surface portion 71 and the second surface portion 72.

As shown in FIG. 26, in the pressurized state, the at least one bulging portion 70 preferably bulges inward in the radial direction y of the outer tube 10, to close a portion of the lumen 11 of the outer tube 10. By closing a portion of the lumen 11, a member (for example, inner tube 50 or guide wire 2) disposed inside the lumen 11 can be gripped. In the same manner as in the example in FIG. 25, movement of, for example, the guide wire 2 in the longitudinal direction x is easily restricted, whereby the inner tube 50 is easily removed without removing the guide wire 2 from the lumen of the outer tube 10.

A frictional force of a surface of the bulging portion 70 located on an inner side in the radial direction y of the outer tube 10 is preferably higher than a frictional force of an inner surface 10c, of the outer tube 10, that faces the lumen 11 of the outer tube 10. A frictional force of the surface of the bulging portion 70 located on the inner side in the radial direction y of the outer tube 10 in the pressurized state is more preferably higher than a frictional force of the inner surface 10c, of the outer tube 10, that faces the lumen 11 of the outer tube 10 in the pressurized state. The above configuration makes it easy to restrict movement, in the longitudinal direction x, of the member disposed inside the lumen 11 of the outer tube 10. In the case where a portion of the outer tube 10 is formed as the bulging portion 70 as shown in FIGS. 17 to 19, the frictional force of the surface of the bulging portion 70 located on the inner side in the radial direction y of the outer tube 10 is preferably higher than a frictional force of a portion of the inner surface 10c, of the outer tube 10, that faces the lumen 11 of the outer tube 10, the portion being a part of the outer tube 10 other than the bulging portion 70. The frictional force of the surface of the bulging portion 70 located on the inner side in the radial direction y of the outer tube 10 in the pressurized state is more preferably higher than a frictional force of the portion of the inner surface 10c, of the outer tube 10, that faces the lumen 11 of the outer tube 10, the portion being a part of the outer tube 10 other than the bulging portion 70, in the pressurized state.

As shown in FIG. 17, in a state where the inward bulging portion 73 is pressurized at the first predetermined pressure or higher pressure, an angle γ formed between the inner walls of the bag-shaped body 20 in the cross section parallel to the longitudinal direction x of the outer tube 10 is preferably an acute angle. The angle γ may be 85° or less, 80° or less, or 70° or less. Meanwhile, the angle γ may be 1° or more, 3° or more, or 5° or more. With this configuration, the thickness of the bag-shaped body 20 at the distal end portion thereof becomes small. In other words, the length between the inner surface of the inner surface portion 21 and the outer surface of the outer surface portion 22 is easily shortened.

In the case where the catheter 1 has the inward bulging portion 73, the position of the bag-shaped body 20 at which the inner diameter thereof is minimum in the non-pressurized state is preferably the distal end of the bag-shaped body 20 as shown in FIG. 4. A position of the bag-shaped body 20 at which the inner diameter thereof is minimum in a state where pressurization at the second predetermined pressure or higher pressure or at the first predetermined pressure or higher pressure is performed by injecting a fluid into the flow path 30 may be the proximal end of the bag-shaped body 20 or the distal end of the bag-shaped body 20.

In the case where the catheter 1 has the inward bulging portion 73, the position of the bag-shaped body 20 at which the outer diameter thereof is minimum in the non-pressurized state is preferably the distal end of the bag-shaped body 20 as shown in FIG. 4. As shown in FIG. 6, a position of the bag-shaped body 20 at which the outer diameter thereof is minimum in a state where pressurization at the second predetermined pressure or higher pressure is performed by injecting a fluid into the flow path 30 is preferably the distal end of the bag-shaped body 20. As shown in FIG. 17, a position of the bag-shaped body 20 at which the outer diameter thereof is minimum in a state where pressurization at the first predetermined pressure or higher pressure is performed by injecting a fluid into the flow path 30 is preferably the distal end of the bag-shaped body 20.

As shown in FIGS. 20 to 24, the outward bulging portion 74 is inflated so as to project outward as a result of being pressurized by injecting a fluid into the inner space of the outward bulging portion 74. At this time, the outward bulging portion 74 easily comes into contact with the wall of a body cavity, whereby movement of the catheter 1 inside the body cavity can be restricted.

The catheter 1 provided with the outward bulging portion 74 can be used according to, for example, the following procedure. First, the catheter 1 which is in the non-pressurized state as shown in FIGS. 4 and 14 is delivered to a treatment site. As shown in FIG. 20, the outward bulging portion 74 is pressurized so as to project outward. Consequently, the outward bulging portion 74 comes into contact with the wall of a body cavity, whereby movement of the catheter 1 inside the body cavity can be restricted. In the state where the outward bulging portion 74 is caused to bulge, the bag-shaped body 20 is pressurized by injecting a fluid into the flow path 30 as shown in FIGS. 21 and 23. As shown in FIG. 24, the inner tube 50 is caused to project from the outer tube 10.

The second predetermined pressure may be equal to or higher than the first predetermined pressure. Consequently, movement of the outer tube 10 inside a body cavity is easily restricted when a medical implement is caused to project from the bag-shaped body 20.

The second predetermined pressure may be higher than the first predetermined pressure. Consequently, in the case where a flow path for injecting a fluid into the outward bulging portion 74 and the flow path for injecting a fluid into the bag-shaped body 20 are in communication with each other, or in a case where the flow path for injecting a fluid into the outward bulging portion 74 and the flow path for injecting a fluid into the bag-shaped body 20 have a portion shared by each other, the diameter of the bag-shaped body 20 can be increased after the outward bulging portion 74 is caused to bulge. For example, the catheter 1 shown in FIG. 4 in the non-pressurized state is pressurized at the first predetermined pressure or higher pressure by injecting a fluid into the flow path 30 so as to cause the outward bulging portion 74 to bulge as shown in FIG. 20, and then the bag-shaped body 20 is pressurized at the second predetermined pressure (higher than the first predetermined pressure) or higher pressure by further injecting the fluid into the flow path 30 so as to increase the minimum inner diameter of the bag-shaped body 20 as shown in FIG. 21.

The second predetermined pressure may be equal to the first predetermined pressure. Consequently, in a case where the flow path for injecting a fluid into the outward bulging portion 74 and the flow path for injecting a fluid into the bag-shaped body 20 are in communication with each other, or in a case where the flow path for injecting a fluid into the outward bulging portion 74 and the flow path for injecting a fluid into the bag-shaped body 20 have a portion shared by each other, increase of the diameter of the bag-shaped body 20 and bulging of the outward bulging portion 74 can be simultaneously performed. For example, when the catheter 1 shown in FIG. 4 in the non-pressurized state is pressurized at the first predetermined pressure or higher pressure (i.e., at the second predetermined pressure or higher pressure) by injecting a fluid into the flow path 30, the outward bulging portion 74 bulges and the minimum inner diameter of the bag-shaped body 20 increases simultaneously, as shown in FIG. 21.

The second predetermined pressure may be lower than the first predetermined pressure. In this case, a medical implement is preferably caused to project from the bag-shaped body 20 after the bulging portion 70 is caused to bulge.

A frictional force of an outer surface of the at least one outward bulging portion 74 is preferably higher than a frictional force of an outer surface 10d of the outer tube 10. A frictional force of the outer surface of the at least one outward bulging portion 74 in the pressurized state is more preferably higher than a frictional force of the outer surface 10d of the outer tube 10 in the pressurized state. The above configuration makes it easy to restrict movement of the catheter 1 inside a body cavity. In the case where a portion of the outer tube 10 is formed as the outward bulging portion 74 as shown in FIG. 20, the frictional force of the outer surface of the outward bulging portion 74 is preferably higher than a frictional force of a portion of the outer surface 10d of the outer tube 10, the portion being a part of the outer tube 10 other than the outward bulging portion 74. The frictional force of the outer surface of the outward bulging portion 74 in the pressurized state is more preferably higher than a frictional force of the portion of the outer surface 10d of the outer tube 10, the portion being a part of the outer tube 10 other than the outward bulging portion 74, in the pressurized state.

As shown in FIG. 20, in a case where the catheter 1 has the outward bulging portion 74, it is preferable that, in a pressurized state where the outward bulging portion 74 is pressurized at the first predetermined pressure or higher pressure by injecting a fluid into the flow path 30, an angle ε formed between the inner walls of the bag-shaped body 20 in the cross section parallel to the longitudinal direction x of the outer tube 10 is an acute angle. The angle ε may be 85° or less, 80° or less, or 70° or less. Meanwhile, the angle ε may be 1° or more, 3° or more, or 5° or more.

As shown in FIG. 21, in the case where the catheter 1 has the outward bulging portion 74, it is preferable that, in a pressurized state where the bag-shaped body 20 is pressurized at the second predetermined pressure or higher pressure by injecting a fluid into the flow path 30, an angle (formed between the inner walls of the bag-shaped body 20 in the cross section parallel to the longitudinal direction x of the outer tube 10 is an acute angle. The angle (may be 85° or less, 80° or less, or 70° or less. Meanwhile, the angle (may be 1° or more, 3° or more, or 5° or more. With this configuration, the thickness of the bag-shaped body 20 at the distal end portion thereof becomes small. In other words, the length between the inner surface of the inner surface portion 21 and the outer surface of the outer surface portion 22 is easily shortened. Consequently, the inner diameter of the distal end portion of the bag-shaped body 20 is easily increased when pressurization at the second predetermined pressure or higher pressure is performed, and, when a medical implement is delivered to a treatment site through the lumen 11, the medical implement is easily caused to project from the diameter-increased portion of the bag-shaped body 20.

In the case where the catheter 1 has the outward bulging portion 74, the position of the bag-shaped body 20 at which the inner diameter thereof is minimum in the non-pressurized state is preferably the distal end of the bag-shaped body 20 as shown in FIG. 4. In the case where the catheter 1 has the outward bulging portion 74, the position of the bag-shaped body 20 at which the inner diameter thereof is minimum in the state where pressurization at the first predetermined pressure or higher pressure is performed by injecting a fluid into the flow path 30 is preferably the distal end of the bag-shaped body 20 as shown in FIG. 20. The position of the bag-shaped body 20 at which the inner diameter thereof is minimum in the state where pressurization at the second predetermined pressure or higher pressure is performed by injecting a fluid into the flow path 30 may be the proximal end of the bag-shaped body 20 or the distal end of the bag-shaped body 20.

In the case where the catheter 1 has the outward bulging portion 74, the position of the bag-shaped body 20 at which the outer diameter thereof is minimum in the non-pressurized state is preferably the distal end of the bag-shaped body 20 as shown in FIG. 4. As shown in FIG. 20, the position of the bag-shaped body 20 at which the outer diameter thereof is minimum in the state where pressurization at the first predetermined pressure or higher pressure is performed by injecting a fluid into the flow path 30 is preferably the distal end of the bag-shaped body 20. As shown in FIG. 21, the position of the bag-shaped body 20 at which the outer diameter thereof is minimum in the state where pressurization at the second predetermined pressure or higher pressure is performed by injecting a fluid into the flow path 30 is preferably the distal end of the bag-shaped body 20.

As can be understood from FIGS. 17 to 24, the catheter 1 may have at least one inward bulging portion 73 and at least one outward bulging portion 74.

The catheter 1 may further have a radiopaque marker 3 provided to at least one of the bag-shaped body 20 and a region, of the outer tube 10, that extends by 10 cm toward the proximal side from the distal end of the outer tube 10. With this configuration, use of an X-ray imaging device makes it possible to visually recognize the position of a distal end portion of the catheter 1. In FIG. 9, the marker 3 is provided to the outer tube 10. In FIGS. 16, 19, 23, and 24, the marker 3 is provided to the bag-shaped body 20.

The radiopaque marker 3 may be provided merely to the bag-shaped body 20, may be provided merely to the region, of the outer tube 10, that extends by 10 cm toward the proximal side from the distal end 10a of the outer tube 10, or may be provided to both the bag-shaped body 20 and the outer tube 10.

As shown in FIGS. 16, 19, 23, and 24, the marker 3 is preferably provided to the distal end portion of the bag-shaped body 20. Consequently, it can be made easy to visually recognize the proximal end of the expansion member 60 in contact with a treatment site described later.

The shape of the above radiopaque marker is preferably a tubular shape, and examples of the shape include a cylindrical shape, a polygonal tubular shape, a shape obtained by forming a slit in a tube so as to have a C-shaped cross section, and a coil shape obtained by winding a wire material.

Examples of the material for forming the above radiopaque marker can include radiopaque substances such as lead, barium, iodine, tungsten, gold, platinum, iridium, stainless steel, titanium, and a cobalt-chromium alloy. The marker may be formed by dispersing radiopaque particles of barium sulfate or the like in the outer tube 10, the bag-shaped body 20, or a resin member that may be separately provided.

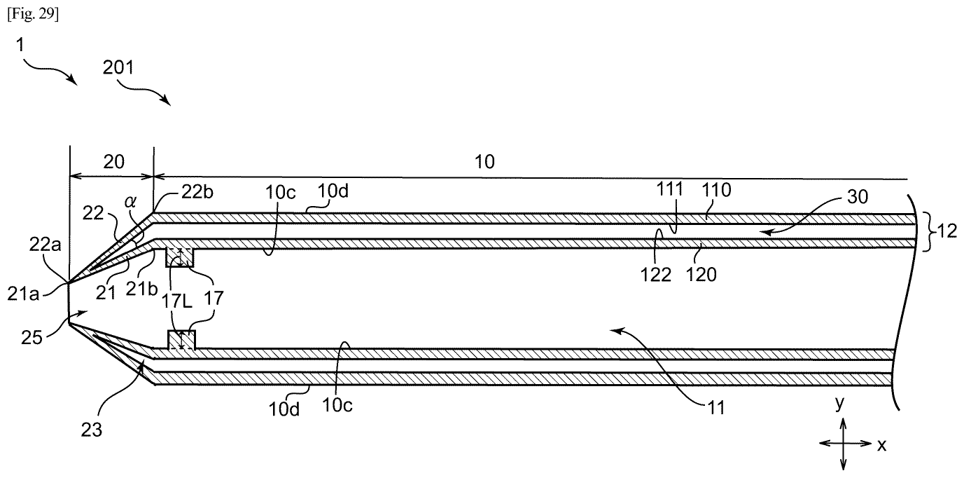

As shown in FIG. 29, the outer tube 10 in the non-pressurized state may have at least one projecting portion 17 projecting inward in the radial direction y of the outer tube 10. In other words, the at least one projecting portion 17 is a portion projecting inward in the radial direction y of the outer tube 10 regardless of whether or not a fluid has been injected into the flow path 30. Consequently, movement, in the longitudinal direction x, of a member disposed inside the lumen of the outer tube 10 can be easily restricted.

The at least one projecting portion 17 may be one projecting portion or a plurality of projecting portions. The plurality of projecting portions are preferably disposed at positions that differ in the circumferential direction on the outer tube 10. The at least one projecting portion 17 may be present over a portion in the circumferential direction of the outer tube 10 or may be present over the entirety in the circumferential direction of the outer tube 10.

As shown in FIG. 29, the at least one projecting portion 17 is preferably disposed on the inner surface of the outer tube 10. For example, the thickness of the inner wall of a portion of the outer tube 10 is made larger than the thickness of the inner wall of the other portion of the outer tube 10, whereby the projecting portion 17 can be formed.

A projection height 17L of the projecting portion 17 and a bulging height 70L of the bulging portion 70 may be equal to or different from each other. The projection height 17L of the projecting portion 17 may be higher than the bulging height 70L of the bulging portion 70. The projection height 17L of the projecting portion 17 may be lower than the bulging height 70L of the bulging portion 70. The projection height 17L of the projecting portion 17 refers to the length of a portion projecting in the radial direction y of the outer tube 10. The bulging height 70L of the bulging portion 70 refers to the length of a portion bulging in the radial direction y of the outer tube 10.

The catheter 1 preferably has a first shaft 201 including: the outer tube 10 having a distal end and a proximal end and having the lumen 11 extending in the longitudinal direction x; the bag-shaped body 20 provided to the distal portion of the outer tube 10, the bag-shaped body 20 having an annular shape and having a portion that has a smaller outer diameter than the outer tube 10; and the flow path 30 which allows a fluid to be injected therethrough to the bag-shaped body 20. A lumen of the first shaft 201 is preferably composed of the lumen 11 of the outer tube 10 and the lumen 25 of the bag-shaped body 20. The flow path 30 may be a flow path that allows a fluid to be injected therethrough to the bag-shaped body 20 and the bulging portion 70.

The catheter 1 preferably has a second shaft 202 disposed inside the lumen 11 of the outer tube 10. The second shaft 202 preferably includes a medical implement such as a balloon, a stent, a basket, or a needle. The second shaft 202 is preferably disposed inside the lumen of the first shaft 201 in a state of being movable relative to the first shaft 201. The second shaft 202 preferably includes: the inner tube 50; and the expansion member 60 which is fixed to the distal portion of the inner tube 50 and which is expanded in the radial direction y.

After a portion of the second shaft 202 is caused to project from the first shaft 201, the following state is preferably obtained as shown in FIGS. 19 and 24, for example. That is, a first portion 63 as a portion of the expansion member 60 is located inside the lumen of the first shaft 201, a second portion 64 as a portion, of the expansion member 60, that is located on the distal side relative to the first portion 63 is located outside of the first shaft 201, the maximum outer diameter of the second portion 64 is larger than the maximum outer diameter of the outer tube 10, and the outer surface of the first portion 63 is inscribed in the first shaft 201. Consequently, the length of the expansion member 60 in contact with a treatment site is easily adjusted to a desired length.

### DESCRIPTION OF THE REFERENCE CHARACTERS

1: catheter
2: guide wire
3: radiopaque marker
10: outer tube
10a: distal end of the outer tube
10b: proximal end of the outer tube
10c: inner surface
10d: outer surface
11: lumen of the outer tube
11a: distal opening
11b: proximal opening
12: wall
13: reduced diameter area
16: reinforcing portion
17: projecting portion
17L: projection height
20: bag-shaped body
21: inner surface portion
21a: distal end of the inner surface portion
21b: proximal end of the inner surface portion
22: outer surface portion
22a: distal end of the outer surface portion
22b: proximal end of the outer surface portion
23: inner space
24: hole
25: lumen of the bag-shaped body
30: flow path
31: second tubular member
40: first tubular member
41: lumen of the first tubular member
42: hub
50: inner tube
51: first inner tube
52: second inner tube
60: expansion member
61: outer surface
63: first portion
64: second portion
70: bulging portion
70L: bulging height
71: first surface portion
72: second surface portion
73, 73A, 73B: inward bulging portion
74, 74A, 74B: outward bulging portion
110: first outer tube
111: inner surface of the first outer tube
120: second outer tube
122: outer surface of the second outer tube
201: first shaft
202: second shaft
x: longitudinal direction of the outer tube
y: radial direction of the outer tube

1. A catheter comprising:
an outer tube having a distal end and a proximal end and having a lumen extending in a longitudinal direction;
a bag-shaped body provided to a distal portion of the outer tube, the bag-shaped body having an annular shape and having a portion that has a smaller outer diameter than the outer tube; and
a flow path configured to allow a fluid to be injected therethrough to the bag-shaped body, wherein
the bag-shaped body is configured so that a minimum inner diameter of the bag-shaped body in a pressurized state where the bag-shaped body is pressurized by injecting a fluid into the flow path is larger than a minimum inner diameter of the bag-shaped body in a non-pressurized state where the bag-shaped body is not pressurized by injecting a fluid into the flow path.

2. The catheter according to claim 1, wherein
the bag-shaped body is further configured so that in the non-pressurized state of the bag-shaped body, the bag-shaped body has an inner surface portion facing an inner side and an outer surface portion facing an outer side, and when a state of the bag-shaped body is changed from the non-pressurized state to the pressurized state, an elongation rate of a length from a distal end to a proximal end of the inner surface portion is higher than an elongation rate of a length from a distal end to a proximal end of the outer surface portion.

3. The catheter according to claim 2, wherein, in at least one of the non-pressurized state of the bag-shaped body and the pressurized state of the bag-shaped body, an angle formed between the inner surface portion and the outer surface portion in a cross section parallel to the longitudinal direction is an acute angle.

4. The catheter according to claim 2, wherein the bag-shaped body and the outer tube are further configured so that when the state of the bag-shaped body is changed from the non-pressurized state to the pressurized state, the elongation rate of the length from the distal end to the proximal end of the inner surface portion is higher than an elongation rate of a length from the distal end to the proximal end of the outer tube.

5. The catheter according to claim 1, wherein the bag-shaped body is disposed on a distal side relative to the distal end of the outer tube and has a tapered portion having an outer diameter that is reduced toward the distal side.

6. The catheter according to claim 1, wherein the catheter further comprises a radiopaque marker, wherein
the radiopaque marker is provided to at least one of the bag-shaped body and a region, of the outer tube, that extends 10 cm toward a proximal side from the distal end of the outer tube.

7. The catheter according to claim 1, wherein the outer tube has, at a proximal portion of the outer tube, a reduced diameter area having a diameter that is reduced toward a proximal side in a cross-sectional view perpendicular to the longitudinal direction.

8. The catheter according to claim 7, wherein
the catheter further comprises a first tubular member having a maximum outer diameter smaller than a maximum outer diameter of the outer tube, the first tubular member having a lumen extending in the longitudinal direction,
the first tubular member has a distal portion fixed to a proximal portion of the reduced diameter area of the outer tube, and
the lumen of the first tubular member and the flow path are in communication with each other.

9. The catheter according to claim 1, wherein, in the pressurized state, the bag-shaped body has an outer diameter equal to or smaller than an outer diameter of the outer tube.

10. The catheter according to claim 1, wherein
the catheter further comprises at least one bulging portion configured to bulge inward in a radial direction of the outer tube or outward in the radial direction of the outer tube, and
the bulging portion and the flow path are configured to allow a fluid to be injected to the bulging portion though the flow path.

11. The catheter according to claim 10, wherein
the at least one bulging portion is configured to bulge inward in the radial direction of the outer tube when the at least one bulging portion is pressurized at a first predetermined pressure or higher pressure by injecting a fluid into the flow path,
the bag-shaped body is configured so that a minimum inner diameter of the bag-shaped body in a state where the bag-shaped body is pressurized at a second predetermined pressure or higher pressure by injecting a fluid into the flow path is larger than the minimum inner diameter of the bag-shaped body in the non-pressurized state, and
the second predetermined pressure is lower than the first predetermined pressure.

12. The catheter according to claim 10, wherein
the at least one bulging portion is configured to bulge outward in the radial direction of the outer tube when the at least one bulging portion enters a state of being pressurized at a first predetermined pressure or higher pressure by injecting a fluid into the flow path,
the bag-shaped body is configured so that a minimum inner diameter of the bag-shaped body in a state where the bag-shaped body is pressurized at a second predetermined pressure or higher pressure by injecting a fluid into the flow path is larger than the minimum inner diameter of the bag-shaped body in the non-pressurized state, and
the second predetermined pressure is equal to or higher than the first predetermined pressure.

13. The catheter according to claim 10, wherein the flow path, the bag-shaped body, and the bulging portion are configured so that a fluid injected is allowed to flow to the bag-shaped body passes through the flow path and the bulging portion.

14. The catheter according to claim 10, wherein
the at least one bulging portion has a first inward bulging portion and a second inward bulging portion configured to bulge inward in the radial direction of the outer tube and arranged at positions that mutually differ in a circumferential direction, and
the first inward bulging portion and the second inward bulging portion are configured so that in the pressurized state, the first inward bulging portion comes into contact with the second inward bulging portion.

15. The catheter according to claim 10, wherein, in the pressurized state, the at least one bulging portion bulges inward in the radial direction of the outer tube, to close a portion of the lumen of the outer tube.

16. The catheter according to claim 10, wherein, in the pressurized state, a bulging height of the at least one bulging portion in the radial direction of the outer tube varies according to a position of the outer tube in a circumferential direction.

17. The catheter according to claim 10, wherein
the at least one bulging portion has a first bulging portion and a second bulging portion arranged at positions that mutually differ in a circumferential direction, and
a bulging height of the first bulging portion and a bulging height of the second bulging portion differ from each other.

18. The catheter according to claim 10, wherein
the at least one bulging portion bulges inward in the radial direction of the outer tube, and
a frictional force of a surface of the at least one bulging portion located on an inner side in the radial direction of the outer tube is higher than a frictional force of an inner surface, of the outer tube, that faces the lumen of the outer tube.

19. The catheter according to claim 10, wherein
the at least one bulging portion bulges outward in the radial direction of the outer tube, and
a frictional force of an outer surface of the at least one bulging portion is higher than a frictional force of an outer surface of the outer tube.

20. The catheter according to claim 10, wherein
the at least one bulging portion has an outward bulging portion configured to bulge outward in the radial direction of the outer tube in the pressurized state, and
the at least one bulging portion is located on a proximal side relative to a proximal end of the bag-shaped body.

21. A catheter comprising:
an outer tube having a distal end and a proximal end and having a lumen extending in a longitudinal direction, the outer tube having a hollow portion at a distal portion, wherein the hollow portion annularly extends within a wall defining the outer tube; and
a flow path configured to allow a fluid to be injected therethrough to the hollow portion, wherein
the outer tube is configured so that a minimum inner diameter at a distal end portion of the outer tube in a pressurized state where the hollow portion is pressurized by injecting a fluid into the flow path is larger than a minimum inner diameter at the distal end portion of the outer tube in a non-pressurized state where the hollow portion is not pressurized by injecting a fluid into the flow path.

22. A catheter comprising:
an outer tube having a distal end and a proximal end and having a lumen extending in a longitudinal direction, the outer tube having a hollow portion at a distal portion, wherein the hollow portion annularly extends within a wall defining the outer tube; and
a flow path configured to allow a fluid to be injected therethrough to the hollow portion, wherein
the outer tube is configured so that an inner diameter of the outer tube at a distal end portion is enlarged by injecting a fluid into the flow path to apply pressure to the follow portion.
